(19) United States
(12) Patent Application Publication
(10) Pub. No.: US 2010/0021928 A1
(43) Pub. Date: Jan. 28, 2010

(54) METHODS AND KITS FOR DIAGNOSING CANCER

(75) Inventors: Rafael M. Nagler, Timrat (IL); Moshe Gavish, Tel-Aviv (IL)

Correspondence Address: MARTIN D. MOYNIHAN d/b/a PRTSI, INC., P.O. BOX 16446, ARLINGTON, VA, 22215, US

(73) Assignee: TECHNION RESEARCH & DEVELOPMENT FOUNDATION LTD., Haifa (IL)

(21) Appl. No.: 12/306,548

(22) PCT Filed: Jun. 25, 2007
(86) PCT No.: PCT/IL2007/000769

§ 371 Date: Sep. 17, 2009

Related U.S. Application Data
(60) Provisional application No. 60/816,312, filed on Jun. 26, 2006.

(51) Int. Cl. G01N 33/53; C12Q 1/28

(52) U.S. Cl. 435/7.1; 435/28

(57) ABSTRACT

Methods and kits for diagnosing cancer in a subject is disclosed. The method comprises determining a level and/or activity of at least one saliva secreted marker in a saliva sample of the subject wherein an alteration in said marker with respect to an unaffected saliva sample is indicative of the cancer, with the proviso that the saliva secreted marker is not circulatory carcinoembryonic antigen (CEA).

### FIELD AND BACKGROUND OF THE INVENTION

Oral squamous cell carcinoma (OSCC) is a common human malignancy, with an increasing incidence (especially in younger people) and a 5-year mortality rate of approximately 50%, which has not changed significantly in more than 50 years. Its location and treatment in the mouth/face/neck result in a relatively high rate of related morbidity, as the treatment frequently results in a significant mutilation and compromised functions. OSCC includes both mobile (oral) and base of tongue cancer lesions. Often, an oral cancer lesion is located at the lateral border of the tongue, whereas one located at the base of tongue is considered especially lethal.

Clinically, it is important to note that the therapeutic modality currently offered to patients is based on traditional stage-predicting indices (based mostly on the tumor-nodemetastasis criteria) and on histologic grading. Unfortunately, these predictors are subjective and relatively unreliable, as often two tumors with identical staging and grading behave in totally different fashions, and although one responds to therapy, the other is lethal. Accordingly, there has been an ever-growing effort dedicated to the basic research of oral cancer, focusing on the identification of biological indicators for the diagnosis of its biological nature and aggressiveness.

Circulatory tumor markers for OSCC were investigated in various studies and showed relatively moderate sensitivity and specificity values with relation to diagnosis, prognosis predicting, or treatment monitoring. For example, Kurokawa et al. analyzed circulatory carcinoembryonic antigen (CEA), SCC, immunosuppressive acidic protein, and Cyfra concentrations in OSCC patients and found sensitivity and accuracy values of 81% and 77.8%, respectively. When CEA, SCC, and immunosuppressive acidic protein were analyzed alone, the values were 69% and 90.3%, respectively [Kurokawa H, et al. Int J Oral Maxillofac Surg 1993;22:35-8; Kurokawa H, et al J Oral Maxillofac Surg 1997;55:964-6].

Hoffmann et al. [Hoffmann J, et al., Intraoperative J Oral Maxillofac Surg 1998;56:1390-3] and Krimmel et al. [Krimmel M et al., J Craniomaxillofac Surg 1998;26:243-8], who analyzed circulatory levels of SCC, CEA, CA19-9, and CA125, found correlation with the tumor burden for only the SCC antigen. They reported rather low sensitivity values for this antigen (except for patients with distant metastasis). They noted that the circulatory SCC antigen had not been routinely used previously, as its reported sensitivity was relatively low in other studies as well (15-40%), although its specificity was quite high (70-90%).

Hellner et al. [Hellner D et al., Dtsch Z Mund Kiefer Gesichtschir 1989;13:291-5. German] reported that circulatory SCC sensitivity in oral cancer patients was only 24%, whereas it was much lower for CEA. Zoller et al. [Zoller J. Dtsch Z Mund Kiefer Gesichtschir 1990;14:254-9; Zoller J. Dtsch Zahn Mund Kieferheilkd Zentralbl 1992;80:351-7. Review. German] reported that, although CA19-9, CA125, and CA15-3 exhibited poor sensitivity, the sensitivity values for circulatory SCC and CEA in oral cancer patients were 33% and 43%, respectively.

Such a wide range was also found for other circulatory markers, such as Cyfra 21-1 or tissue polypeptide antigen (TPS), which were in the range of 25% to 96% and 65% to 75%, respectively [Nagler R M, et al., Cancer 1999;35:1018-25; Tumour Biol 1993;14:55-8; Bhatavdekar J M, et al., Anticancer Res 1993;13:237-40; Yen T C et al., Clin Otolaryngol 1998;23:82-6].

One method suggested, to improve the sensitivity and accuracy of such an analysis, was to examine various circulatory markers concurrently (“combination assay” [Kurokawa H, et al., Int J Oral Maxillofac Surg 1993;22:35-8; Kurokawa H, et al., J Oral Maxillofac Surg 1997;55:964-6]).

Negri L. et al and Airoldi M. et al both teach examination of CEA in the saliva for the detection of OSCC [Negri L, et al., Int J Biol Markers 1988, 3:107-12; Airoldi M, et al., Boll Soc Ital Biol Sper 1984, 60:865-70]. This test proved to be both non-specific and insensitive.

Free radicals, such as reactive oxygen and nitrogen species (ROS and RNS), which induce oxidative and nitrative stress, are principal inducers of OSCC. Ma et al [Nitric Oxide. 2006;14:137-143] recently demonstrated that oxidative and nitrative stress contribute to the development of oral carcinogenesis from leukoplakia through DNA damage. RNS in the form of nitrosamines (NO₃ and NO₂) and ROS such as superoxide radicals (O₂⁻), hydroxyl radicals (OH⁻), and hydrogen peroxide (H₂O₂), play a key role in human cancer development because they can cause DNA base alterations, strand breaks, damaged tumor suppressor genes, and an enhanced expression of protooncogenes. ROS-induced mutation could also result from protein damage.

Salivary nitrosamine production and metabolism are also based on the dietary nitrates (NO₃), which are absorbed from the upper gastrointestinal tract and actively concentrated from the plasma into the saliva by the salivary glands through an active transport system similar to that for iodide, thiocyanate, and perchlorate. In the oral cavity the salivary nitrates are turned into nitrites (NO₂), which are of special importance as carcinogenesis promoters because they react with amines and amides to form the carcinogenic nitrosamines.

The OSCC-inducing ROS and RNS originate mainly from smoking, alcohol, food, drink, and/or various other volatile sources, which enter freely into the oral cavity through the largest open gate of the body, the mouth. The salivary antioxidant system is based on enzymatic and non-enzymatic components including peroxidase and superoxide dismutase (SOD) enzymes as well as uric acid (UA) molecules. It also includes another pivotal anticancer salivary enzyme, glutathione S-transferase (GST), which catalyzes glutathione conjugation to the carcinogen electrophilic epoxide intermediates to protect against DNA damage and adduct formation.

U.S. Pat. No. 20040181344 teaches diagnosis of oral cancer by analyzing an expression profile of a particular set of polypeptides in a biological sample such as saliva. U.S. Pat. No. 20040181344 does not teach diagnosis of oral cancer by analyzing oxidative stress-related parameters and the antioxidant profile of the saliva.

Li et al also teach salivary transcriptome diagnostics for oral cancer detection [Li et al., Clinical Cancer Research Vol. 10, 8442-8450, Dec. 15, 2004]. Specifically, Li et al teach that transcripts of IL8, IL1B, DUSP1, HA3, OAZ1, S100P, and SAT may serve as potential salivary RNA biomarkers.

Of note, neither U.S. Pat. No. 20040181344 nor Li et al teach analysis of saliva secreted markers. Instead, the expression profile of cellular proteins is analyzed.

There remains a widely recognized need for, and it would be highly advantageous to have other accurate and sensitive methods for detecting OSCC.

### SUMMARY OF THE INVENTION

According to one aspect of the present invention there is provided a method of diagnosing cancer in a subject, the method comprising determining a level and/or activity of at least one saliva secreted marker in a saliva sample of the subject wherein an alteration in the marker with respect to an unaffected saliva sample is indicative of the cancer, with the proviso that the saliva secreted marker is not circulatory carcinoembryonic antigen (CEA).

According to another aspect of the present invention there is provided a method of diagnosing cancer in a subject, the method comprising determining a level and/or activity of at least one marker in a saliva sample of the subject wherein an alteration in the marker with respect to an unaffected saliva sample is indicative of the cancer, wherein the saliva marker is selected from the group consisting of tissue polypeptide-specific antigen (TPS), Cyfra 21-1, 8-Hydroxy-2′-deoxyguanosine (8OHDG), Squamous cell carcinoma (SCC) antigen, CA19-9, CA125, a free radical, a nitrate, a nitrite, a nitric oxide, a carbonyl polypeptide, a thiobarbituric acid reactive substance (TBARS), malondialdehyde (MDA), glutathione S-transferase (GST), Superoxide dismutase (SOD), Uric acid (UA), Ferrylmyoglobin, total antioxidant status (TAS), peroxidase, antioxidant capacity (ImAnOx), Metalloproteinase, Benzodiazepine receptor, pH, Heparanase, total protein, amylase, an electrolyte, lactate dehydrogenase (LDH), insulin-like growth factor (IGF), epidermal growth factor (EGF) and albumin.

According to yet another aspect of the present invention there is provided a kit for diagnosing cancer in a subject, the kit comprising a packaging material which comprises at least one agent for specifically determining a level and/or activity of at least one saliva secreted marker in a saliva sample of the subject, with the proviso that the saliva secreted marker is not CEA.

According to still another aspect of the present invention there is provided kit for diagnosing cancer in a subject, the kit comprising a packaging material which comprises at least one agent for specifically determining a level and/or activity of at least one saliva marker in a saliva sample of the subject, the saliva marker being selected from the group consisting of tissue polypeptide-specific antigen (TPS), Cyfra 21-1, 8-Hydroxy-2′-deoxyguanosine (8OHDG), Squamous cell carcinoma (SCC) antigen, CA19-9, CA125, a free radical, a nitrate, a nitrite, a nitric oxide, a carbonyl polypeptide, a thiobarbituric acid reactive substance (TBARS), malondialdehyde (MDA), glutathione S-transferase (GST), Superoxide dismutase (SOD), Uric acid (UA), Ferrylmyoglobin, total antioxidant status (TAS), peroxidase, antioxidant capacity (ImAnOx), Metalloproteinase, Benzodiazepine receptor, pH, Heparanase, total protein, amylase, an electrolyte, lactate dehydrogenase (LDH), insulin-like growth factor (IGF), epidermal growth factor (EGF) and albumin.

According to an additional aspect of the present invention there is provided a device for diagnosing cancer, the device comprising a support and at least one agent for specifically determining a level and/or activity of at least one saliva marker in a saliva sample of the subject attached to the support, the saliva marker being selected from the group consisting of tissue polypeptide-specific antigen (TPS), Cyfra 21-1, 8-Hydroxy-2′-deoxyguanosine (8OHDG), Squamous cell carcinoma (SCC) antigen, CA19-9, CA125, a free radical, a nitrate, a nitrite, a nitric oxide, a carbonyl polypeptide, a thiobarbituric acid reactive substance (TBARS), malondialdehyde (MDA), glutathione S-transferase (GST), Superoxide dismutase (SOD), Uric acid (UA), Ferrylmyoglobin, total antioxidant status (TAS), peroxidase, antioxidant capacity (ImAnOx), Metalloproteinase, Benzodiazepine receptor, pH, Heparanase, total protein, amylase, an electrolyte, lactate dehydrogenase (LDH), insulin-like growth factor (IGF), epidermal growth factor (EGF) and albumin.

According to further features in preferred embodiments of the invention described below, the saliva secreted marker is selected from the group consisting of TPS, Cyfra 21-1, Squamous cell carcinoma (SCC) antigen, CA19-9, CA125, a free radical, a nitrate, a nitrite, a nitric oxide, a carbonyl polypeptide, a thiobarbituric acid reactive substance (TBARS), malondialdehyde (MDA), glutathione S-transferase (GST), Superoxide dismutase (SOD), 8-Hydroxy-2′-deoxyguanosine (8OHDG), Uric acid, Ferrylmyoglobin, peroxidase, Metalloproteinase, Benzodiazepine receptor, Heparanase, total protein, amylase, an electrolyte, lactate dehydrogenase (LDH), insulin-like growth factor (IGF), epidermal growth factor (EGF) and albumin.

According to still further features in the described preferred embodiments, the saliva secreted marker is selected from the group consisting of a tumor marker, a reactive nitrogen species, a reactive oxygen species and an antioxidant marker.

According to still further features in the described preferred embodiments, the reactive oxygen species is selected from the group consisting of a superoxide radical (O₂⁻), a hydroxyl radical (OH⁻), and hydrogen peroxide (H₂O₂).

According to still further features in the described preferred embodiments, the reactive nitrogen species is selected from the group consisting of a nitrate, a nitrite and nitric oxide.

According to still further features in the described preferred embodiments, the tumor marker is selected from the group consisting of TPS, Cyfra 21-1, SCC, CA19-9 and CA125.

According to still further features in the described preferred embodiments, the saliva secreted marker is a polypeptide or a fatty acid.

According to still further features in the described preferred embodiments, the polypeptide is a carbonyl polypeptide.

According to still further features in the described preferred embodiments, the fatty acid is MDA or TBARS.

According to still further features in the described preferred embodiments, the antioxidant marker is selected from the group consisting of GST, SOD, Uric acid, ferrylmyoglobin and a peroxidase.

According to still further features in the described preferred embodiments, the method further comprises determining a level of CEA in the saliva sample.

According to still further features in the described preferred embodiments, the cancer is oral cancer or oral-pharyngeal cancer.

According to still further features in the described preferred embodiments, the at least one agent is an antibody.

According to still further features in the described preferred embodiments, the device is a lateral flow device.

According to still further features in the described preferred embodiments, the device is a dipstick or a cartridge.

The present invention successfully addresses the shortcomings of the presently known configurations by providing a method of diagnosing cancer based on detection of saliva secreted markers.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a bar graph depicting salivary concentrations of CA125, TPS, Cyfra 21-1, CA19-9, CEA, and SCC tumor markers in healthy (empty columns; n=16) and OSCC patients (dotted columns; n=14). The medians of the healthy controls and the OSCC patients were compared with the Wilcoxon rank-sum test (pairs of subgroups). *, P≦0.05; **, P≦0.01.

FIG. 2 is a scatter plot of the salivary CA125 concentrations of examined cancer and healthy subjects.

FIG. 3 is a scatter plot of the salivary TPS concentrations of examined cancer and healthy subjects.

FIG. 4 is a scatter plot of the salivary Cyfra 21-1 concentrations of examined cancer and healthy subjects.

FIG. 5 is a graph depicting overall cumulative survival and DFS probabilities for the 21 tongue SCC patients.

FIG. 6 is a bar graph comparing salivary concentrations of general antioxidants: total antioxidant status (TAS) and antioxidant capacity (ImAnOx) and of oxidized DNA (8-OHdG) in healthy (open bars, n=25) and oral squamous cell carcinoma (OSCC) patients (dotted bars, n=25). Statistical significance: *P≦0.05.

FIG. 7 is a bar graph comparing salivary concentrations of specific antioxidants: peroxidase, glutathione S-transferase (GST), uric acid (UA) and activity levels of superoxide dismutase (SOD) in healthy (open bars, n=25) and oral squamous cell carcinoma (OSCC) patients (dotted bars, n=25). Statistical significance: *P≦0.05.

FIG. 8. is a scatter plot depicting the spearman correlation coefficient between salivary general antioxidant assays: total antioxidant status (TAS) and antioxidant capacity (ImAnOx).

FIG. 9 is a bar graph comparing salivary concentrations of total nitric oxide (NO), nitrates (NO₃) and nitrites (NO₂) in healthy (open bars, n=25) and oral squamous cell carcinoma (OSCC) patients (dotted bars, n=25). Statistical significance: *P≦0.05.

FIG. 10 is a Western blot analysis with anti-DNP antibody for salivary protein carbonylation (oxidation level). Lanes 7-12 (right panel) show the increased level of protein carbonyls in oral squamous cell carcinoma (OSCC) patients as compared with the saliva secreted in the healthy controls (lanes 1-6, left panel).

FIG. 11 is a bar graph comparing salivary concentrations of immunoglobulins, secretory IgA (Sec. IgA) and total IgG, albumin (Alb) and lactate dehydrogenase (LDH) in healthy controls (empty bars, n=25) and OSCC patients (dotted bars, n=25). Median (50 percentile), lower and upper quartile (25 and 75 percentile, respectively), statistical significance: ** P≦0.01.

FIG. 12 is a bar graph comparing salivary concentrations of specific cancer-related cytokines, IGF and EGF and metalloproteases, MMP-2 and MMP-9, in healthy controls (empty bars, n=25) and OSCC patients (dotted bars, n=25). Median (50 percentile), lower and upper quartile (25 and 75 percentile, respectively), statistical significance: * P≦0.05, ** P≦0.01.

FIG. 13 shows an embodiment of the device of the present invention as seen in a longitudinal section.

### DESCRIPTION OF THE PREFERRED EMBODIMENTS

The present invention is of a method of diagnosing oral cancer using patient salivary samples.

The principles and operation of the diagnostic method according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Primary tumors can be identified in bodily fluids tested from affected patients. For example, cancer-related nucleic acids in blood, urine, and cerebrospinal fluid have been used as biomarkers for cancer diagnosis. More recently, mRNA biomarkers in serum or plasma have been targets for reverse transcription-PCR (RT-PCR)-based detection strategies in patients with cancers.

The present inventors rationalized that analysis of the saliva of oral cancer patients may be of great benefit because of the direct contact between the saliva and the cancer lesion. Moreover, using saliva as a diagnostic fluid meets the demands for inexpensive, noninvasive, and accessible diagnostic methodology.

Whilst reducing the present invention to practice, the present inventors uncovered a group of salivary biomarkers in oral cancer patients which serve as accurate predictors of the disease. Furthermore, the present inventors showed that concurrent analysis of a combination of these markers significantly increased the diagnostic accuracy of the test to a clinically acceptable level. Since it is known that salivary analysis is a useful diagnostic tool for other distant malignancies, such as breast carcinoma [Bigler et al., J Oral Pathol Med, 2002;31:421-31], the present inventors envision that the current set of biomarkers may also be used to detect other cancers.

Thus, according to one aspect of the present invention, there is provided a method of diagnosing cancer in a subject, the method comprising determining a level and/or activity of at least one saliva secreted marker in a saliva sample of the subject, wherein an alteration in said marker with respect to an unaffected saliva sample is indicative of the cancer.

As used herein, the term “diagnosing” refers to determining the presence of a cancer, classifying a cancer, determining a severity of cancer (grade or stage), monitoring cancer progression, forecasting an outcome of the cancer and/or prospects of recovery.

The subject may be a healthy animal or human subject undergoing a routine well-being check up. Alternatively, the subject may be at risk of having cancer (e.g., a genetically predisposed subject, a subject with medical and/or family history of cancer, a subject who has been exposed to carcinogens, occupational hazard, environmental hazard] and/or a subject who exhibits suspicious clinical signs of cancer [e.g., blood in the stool or melena, unexplained pain, sweating, unexplained fever, unexplained loss of weight up to anorexia, changes in bowel habits (constipation and/or diarrhea), tenesmus (sense of incomplete defecation, for rectal cancer specifically), anemia and/or general weakness). According to another embodiment, the subject may be a diagnosed cancer patient and is performing a routine check-up, in-between treatments.

The term “cancer” as used herein, refers to a disease or disorder resulting from the proliferation of oncogenically transformed cells. Examples of particular cancers that may be diagnosed according to the method of the present invention include oral cancer, such as oral squamous cell carcinoma and oral pharyngeal cancer.

As used herein, the term “saliva” refers to the oral fluid typically made up of a combination of secretions from a number of sources (e.g., parotid, submandibular, sublingual, accessory glands, gingival mucosa and buccal mucosa).

The saliva analyzed according to the method of the present invention may be stimulated (e.g. by chewing on a piece of paraffin film or tart candy) or unstimulated. According to a preferred embodiment of this aspect of the present invention, the saliva is unstimulated.

Saliva specimens for testing can be collected following various methods known in the art. Proper conditions for generating unstimulated saliva have been described. (Nazaresh and Christiansen, J. Dent. Res. 61: 1158-1162 (1982)). Methods and devices for collecting saliva have also been described. (See also, U.S. Pat. No. 5,910,122 to D'Angelo; U.S. Pat. No. 5,714,341 to Thieme et al.; U.S. Pat. Nos. 5,335,673 and 5,103,836 to Goldstein et al.; U.S. Pat. No. 5,268,148 to Seymour; and U.S. Pat. No. 4,768,238 to Kleinberg et al., incorporated herein in their entirety by reference).

The saliva may be analyzed immediately following collection of the sample. Alternatively, salivary analysis according to the method of the present invention can be performed on a stored saliva sample. The saliva sample for testing can be preserved using methods and apparatuses known in the art. (See e.g., U.S. Pat. No. 5,968,746 to Schneider, hereby incorporated in its entirety by reference). The present invention also contemplates treatment of the saliva prior to analysis (for example, to reduce viscosity and to remove cellular material). Techniques used to remove debris include centrifugation and filtration. The viscosity of saliva can also be reduced by mixing a saliva sample with a cationic quaternary ammonium reagent. (See, U.S. Pat. No. 5,112,758 to Fellman et al., incorporated herein in its entirety by reference).

As used herein, the phrase “saliva secreted marker” refers to a component that is secreted into the saliva (i.e. it does not require cell lysis for detection). The saliva secreted marker may be a polypeptide, such as a tumor marker or a carbonyl polypeptide. Examples of known tumor markers that may be analyzed according to the method of the present invention include, but are not limited to tissue polypeptide-specific antigen (TPS), Cyfra 21-1, Squamous cell carcinoma (SCC) antigen, CA19-9, circulatory carcinoembryonic antigen (CEA) and CA125.

Other polypeptides that may be analyzed according to the method of the present invention include, but are not limited to the antioxidant markers, (e.g. glutathione S-transferase (GST), Superoxide dismutase (SOD), ferrylmyoglobin and peroxidase); Metalloproteinase (e.g. Metalloproteinase 2 or Metalloproteinase 9); Benzodiazepine receptor or subunits thereof; Heparanase; amylase; lactate dehydrogenase (LDH); insulin-like growth factor (IGF); epidermal growth factor (EGF) and albumin.

The present inventors have also shown that measurement of total protein content secreted in the saliva may also be used as a gauge to diagnose cancer. Methods of determining total protein content are known in the art such as by Bradford assay, Lowry assay, OD analysis and the like.

Expression and/or activity level of particular proteins secreted in the saliva can be determined using methods known in the arts.

Enzyme linked immunosorbent assay (ELISA): This method involves fixation of saliva containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Radio-immunoassay (RIA): In one version, this method involves precipitation of the desired protein (i.e., the substrate) with a specific antibody and radiolabeled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical analysis: This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

In situ activity assay: According to this method, a chromogenic substrate is applied on the cells containing an active enzyme and the enzyme catalyzes a reaction in which the substrate is decomposed to produce a chromogenic product visible by a light or a fluorescent microscope.

In vitro activity assays: In these methods the activity of a particular enzyme is measured in a protein mixture extracted from the cells. The activity can be measured in a spectrophotometer well using colorimetric methods or can be measured in a non-denaturing acrylamide gel (i.e., activity gel). Following electrophoresis the gel is soaked in a solution containing a substrate and colorimetric reagents. The resulting stained band corresponds to the enzymatic activity of the protein of interest. If well calibrated and within the linear range of response, the amount of enzyme present in the sample is proportional to the amount of color produced. An enzyme standard is generally employed to improve quantitative accuracy.

Exemplary antibodies and assays that may be used to detect the polypeptide markers of the present invention are further described in the Examples section herein below.

Other saliva secreted markers contemplated for use as diagnostic markers include reactive nitrogen species (RNS) markers, reactive oxygen species (ROS) markers and antioxidant markers. RNS and ROS are principal induces of OSCC and the salivary antioxidant system comprises pivotal anticancer enzymes such as glutathione S-transferase (GST), which catalyzes glutathione conjugation to the carcinogen electrophilic epoxide intermediates to protect against DNA damage and adduct formation.

As used herein, the phrase “reactive nitrogen species marker” refers to a molecule whose presence correlates with the reactive nitrogen species in the saliva. The reactive nitrogen species marker may be a reactive nitrogen species itself or a molecule that is regulated by a reactive nitrogen species. RNS is a nitrogen containing molecule, highly reactive due to the presence of unpaired valence shell electrons. Examples of reactive nitrogen species markers include nitrates, nitrites and nitric oxide. Methods of detecting reactive nitrogen species markers are described in Example 2 of the Examples section herein below.

As used herein, the phrase “reactive oxygen species (ROS) marker” refers to a molecule whose presence correlates with the reactive oxygen species in the saliva. The reactive nitrogen species marker may be a reactive oxygen species itself or a molecule that is regulated by a reactive oxygen species. ROS refers to both inorganic and organic oxygen containing molecules, highly reactive due to the presence of unpaired valence shell electrons, formed as a natural byproduct of the normal metabolism of oxygen. Examples of reactive oxygen species include but are not limited to superoxide radicals (O₂⁻), hydroxyl radicals (OH⁻), and hydrogen peroxide (H₂O₂). Methods of detecting reactive oxygen species markers are described in Example 2 of the Examples section herein below and further described in the Invitrogen handbook section 18.2, “Generating and Detecting Reactive Oxygen Species”.

The phrase “antioxidant marker” as used herein, refers to a molecule whose presence correlates with the amount of antioxidant in the saliva. The antioxidant marker may be an antioxidant itself or a molecule that is regulated by an antioxidant. Examples of antioxidant markers include, but are not limited to Glutathione S-transferase (GST), Superoxide dismutase (SOD), 8-Hydroxy-2′-deoxyguanosine (8OHDG), Uric acid, ferrylmyoglobin and peroxidase. Methods of detecting antioxidant markers are described in Example 2 of the Examples section herein below.

The saliva secreted marker of the present invention may also be a fatty acid such as a thiobarbituric acid reactive substance (TBARS) or malondialdehyde (MDA). Methods of detecting TBARS/MDA are described in Example 2 of the Example section herein below.

In addition the saliva secreted marker of the present invention may be a carbohydrate such as a gloycomine.

The present inventors have shown that electrolytes may also serve as salivary cancer markers (see Example 3). Exemplary electrolytes that may be analyzed according to the method of the present invention include sodium, potassium, calcium, phosphorus and magnesium.

It will be appreciated that the present invention also contemplates salivary characteristics as a gauge for cancer diagnosis. Such salivary characteristics include pH, total antioxidant status (TAS) and antioxidant capacity (ImAnOx).

The term “TAS” as used herein refers to the sum of all the antioxidants in the salivary antioxidants. The antioxidants present in the saliva typically may be divided into three systems as follows:

Primary antioxidants (work by preventing the formation of new free radical species). These include superoxide dismutase (SOD), glutathione peroxidase (GPx) and metal-binding proteins (e.g. ferritin or ceruloplasmin).

Secondary antioxidants (act as trap radicals thereby preventing chain reactions). Examples include Vitamin E, vitamin C, beta-carotene, uric acid, bilirubin, and albumin.

Tertiary antioxidants (repair biomolecules damaged by free radicals). These include DNA repair enzymes.

Thus measurement of TAS typically involves measuring the total amount of primary, secondary and tertiary antioxidants.

The phrase “antioxidant capacity” as used herein, refers to an integrated measurement of the cumulative action of all antioxidants that are present in the saliva, rather than the simple sum of measurable antioxidants.

Methods of measuring TAS and antioxidant capacity are described in Example 2, herein below.

It will be appreciated that a combination of the markers of the present invention may be analyzed in order to diagnose the subject. Accordingly, the present invention anticipates analysis of two markers, three markers, four markers, five markers and six or more markers. According to one embodiment, the markers analyzed for the diagnosis of the cancer include Cyfra 21-1, TPS and CA125, wherein an up-regulation of all three is indicative of the cancer.

As mentioned, the method of the present invention comprises measuring a feature or a component of the saliva and comparing the measurement with an unaffected saliva sample wherein a change in the amount of the salivary component or feature is indicative of the cancer.

As used herein, the phrase “unaffected saliva sample” refers to a saliva sample taken from a healthy subject or from the same subject prior to the onset of the cancer. Since saliva characteristics and quantities of saliva components depend on, amongst other things, species and age, it is preferable that the non-cancerous control saliva come from a subject of the same species, age and from the same sub-population (e.g. smoker/nonsmoker). Alternatively, control data may be taken from databases and literature. It will be appreciated that the control sample may also be taken from the diseased subject at a particular time-point, in order to analyze the progression of the disease.

The term “change” as used herein refers to an up-regulation or a down-regulation.

It will be appreciated that the tools necessary for detecting the salivary markers of the present invention may be provided as a kit, such as an FDA-approved kit, which may contain one or more unit dosage form containing the active ingredient for detection of a salivary marker of the present invention.

Alternatively, the kit may comprise means for collecting the sample and specific antibodies packaged separately.

The kit may be accompanied by instructions for administration. The kit may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration.

For example, the kit may be comprised in a device such as a dipstick or a cartridge, (optionally comprised in a housing) which the subject places into the mouth and detects a change in a salivary component. The device may comprise any agent capable of specifically detecting the salivary markers of the present invention. For example, the device may comprise one or a combination of monoclonal and polyclonal antibody reagents and an indicator for detecting binding. Antibody supports are known in the art. In an embodiment of this invention, antibody supports are absorbent pads to which the antibodies are removably or fixedly attached.

According to a preferred embodiment, the device is a lateral flow device comprising inlet means for flowing saliva into contact with the agents capable of detecting the saliva markers of the present invention. The test device can also include a flow control means for assuring that the test is properly operating. Such flow control means can include control antigens bound to a support which capture detection antibodies as a means of confirming proper flow of sample fluid through the test device. Alternatively, the flow control means can include capture antibodies in the control region which capture the detection antibodies, again indicating that proper flow is taking place within the device.

In one embodiment, the kit comprises a monoclonal biomarker colored conjugate and polyclonal anti-biomarker coated on a membrane test area. By capillary action, the saliva sample migrates over the test area and reacts with the impregnated reagents to form visible colored bands in the test window. The presence of the biomarker in concentrations above normal will result in the formation of a distinct colored band in the test area thus indicating a positive result for the caner. Conversely, if no line appears in the test area, the test is negative.

Reference is now made to FIG. 13, which is a schematic illustration of a device 10, according to various exemplary embodiments of the present invention. Device 10 comprises a solid support 12, which is comprised on one end of an inlet 14 comprising an absorbent material able to draw saliva by capillary. Examples of hydrophylic capillary materials that may be used in accordance with the present invention are paper, cellulose powder cotton or other cellulose derivatives, hydrophylic polymers, polysaccharides or polyols, kaolin, titanium dioxide, barium sulfate, and diatomaceous earth. One side of the inlet 14 is placed in the mouth. The other side of the inlet 14 is attached to a test area 16. The test area 16 is comprised of a membrane or filter which binds agent 18, made from materials such as nitrocellulose, nylon, Immunodyne, Biodyne, activated paper with pore size ranging from 0.45 to 12 μm, Agent 18 may be any agent that is capable of detecting the markers of the present invention. In one embodiment agent 18 is an antibody. It will be appreciated that more than one agent 18 may be fixed on the test area 16. The number of agents 18 fixed on the test area 16 will vary according to the number of markers to be detected.

A flow indicator 20 may be present on the test area 16 and may be, for instance, a pH indicator compound able to change color when wetted by saliva, for example bromophenol blue.

The test area 16 and the absorbent material of the inlet 14 may be sealed in a housing 22 wherein the upper part of the inlet 14 is left free. The device of the invention can be shaped in several forms suited for the intended use, for instance as a stick, small tube, strip-supported on plastic material, paper or the like.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### Examples

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Analysis of Salivary Tumor Markers for the Detection of OSCC

Materials and Methods

Patients and study design: 21 patients who received definitive treatment for tongue SCC were monitored for up to 42 months. The group's mean age was 68±17 (range 30-86) and included 12 females and nine males. For 14 of these patients salivary analysis was obtained as well, which was compared to a control group of 16 healthy individuals matched for age and sex. The data obtained included staging (according to the TNM criteria), histological grading, depth of the tumor, maximal tumor diameter, localization at the base vs. mobile part of the tongue and the patients' age and sex. Other data obtained were the salivary concentrations of the carbohydrate antigens CA125 and CA19-9, tissue polypeptide antigen (TPS), carcinoembryonic antigen (CEA), squamous cell carcinoma antigen (SCC) and Cyfra 21-1. They were measured shortly prior to the administration of the definitive curative treatment, which included surgical removal of the primary tongue tumor, neck dissection and often post-operative adjuvant radiotherapy. These data were correlated with the patients' accumulative survival and disease-free survival data.

Saliva collection: Whole saliva was collected shortly prior to the administration of definitive therapy under non-stimulatory conditions in a quiet room between 8 A.M. and noon, at least one hour after eating. Patients were asked to generate saliva and to spit into a wide test tube for ten minutes as previously described [Hansis E et al., Int J. Biochem Cell Biol 2004;36:826-39]. Following collection, the saliva was immediately centrifuged at 800 g at 4° C. for ten minutes to remove squamous cells and cell debris. The resulting supernatant was used for further biochemical analysis.

Assessment of salivary tumor markers: Salivary samples were stored at −70° C. until analyzed, when all 6 markers were assayed. The TPS and Cyfra 21-1 were analyzed as previously described [Nagler et al., Cancer 1999;35:1018-25; Rydlander et al., Eur J Biochem 1996;241 :309-14]. Briefly, TPS was assayed using the monoclonal immunoradiometric assay (IRMA) of BEKI Diagnostics AB (Sweden). The assay measures the M3 epitope soluble fragments of human cytokeratin 18. Cyfra 21-1 was evaluated using a kit (Elsa-Cyfra 21-1 IRMA kit; CIS Bio-International, Gif-Sur-Yvette, France). Cyfra 21-1 was developed using two monoclonal antibodies (BM 19-21 and KS 19-1) that react with different epitopes on cytokeratin 19 found in the samples. The first monoclonal antibody was immobilized in plastic tubes, whereas the second antibody was iodinated. When the sample contained cytokeratin 19 fragments, their epitopes cross-linked both antibodies, resulting in an increase in the radioactivity as measured by a gamma counter. SCC, CEA, CA 19-9 and CA 125 were determined with a microparticle enzyme-linked immuno-assay (MEIA) distributed by Abbot (Abbot Japan CO., LTD, 1-9-9, Roppongi, Minato-Ku, Tokyo) and performed as previously described [Beretta E, et al. Cancer 1987;60:2428-31; Bast R C Jr, et al. N Engl J Med 1983;309:8837; Staab H J, et al., Cancer Detect Prev 1983;6(1-2): 149-53].

Statistical analysis: For categorical variables, frequencies, percentages and distribution were calculated. For continuous variables ranges, medians, means and standard errors were calculated. Median values were calculated because of the large in-borne variability of parameters in saliva (a common practice). Since small (less than 30) groups were analyzed, non-parametric statistical tests were used. Distributions of categorical variables were compared and analyzed with the Fisher-Irwin exact test. The medians between subgroups of patients were compared with the Wilcoxon rank-sum test (pairs of subgroups). A correlation matrix of estimators was used to analyze the correlation coefficients between the salivary markers. For classification analysis, cutoff values were calculated as mean plus standard error of healthy controls. Sensitivity and specificity values were calculated as the fraction of observations which were correctly classified. The cumulative incidence estimate was used to calculate the probability of survival and disease free survival rates as a function of time. The log-rank test was used to compare pairs of cumulative incidence estimators.

Results

Clinical data, staging, pathological grading, dimensions, site and extension of the tumors: The distribution of the 21 patients according to tumor size (T) revealed that nine had T1 and ten patients had T2 tumors while only two patients had T3 and T4 tumors (one of each). That is, 90% of the patients had early (small to moderate) tumors. In 16/21 (76%) of the patients there were no neck metastasis (N0) while 4 patients were diagnosed with N1 and one with N2. None had distant metastasis (all patients were M0). Accordingly, 71% of the patients were diagnosed with early stage tumors (1+2) while only 29% were diagnosed with advanced stages (3+4). Similarly, most of the patients (80%) were diagnosed with well- and moderately-differentiated tumors (seven and 12 patients with grades 1 and 2, respectively) and only two patients were diagnosed with poorly differentiated lesions

The mean tumor diameter was 2.5±1.3 cm (range 0.8-6.0 cm) and mean depth was 8.5±6.4 mm (range 1-26 mm). The correlation rates between the diameter and T and the diameter and N were 0.82 and 0.25 respectively, while between the depth and T and the depth at N they were 0.40 and 0.34, respectively.

Only 21% of the patients smoked (three of the 14 for whom this information was available). The rate of smokers in the control group was not significantly different (4/16). About 20% of the patients had other or previous malignancies (four of 20 for whom these data were available) but not in the head and neck region and none had previously been treated with radiotherapy. None of the controls was treated with radiotherapy or had previous head and neck cancer. In 17 patients (85%) the tumor was located in the oral (mobile) tongue (oral cancer) (13 in the anterior/middle portion and four tumors located posterior-laterally) while in three patients (15%) it was located at the base of tongue (oropharyngeal cancer). In 26% of the patients (five of 19 available) the tumor extended beyond the lingual region and expanded locally towards neighboring regions, such as the floor of the mouth.

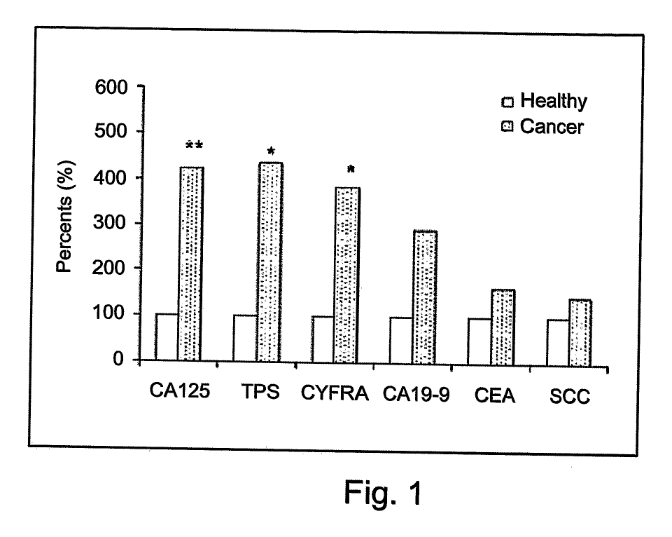

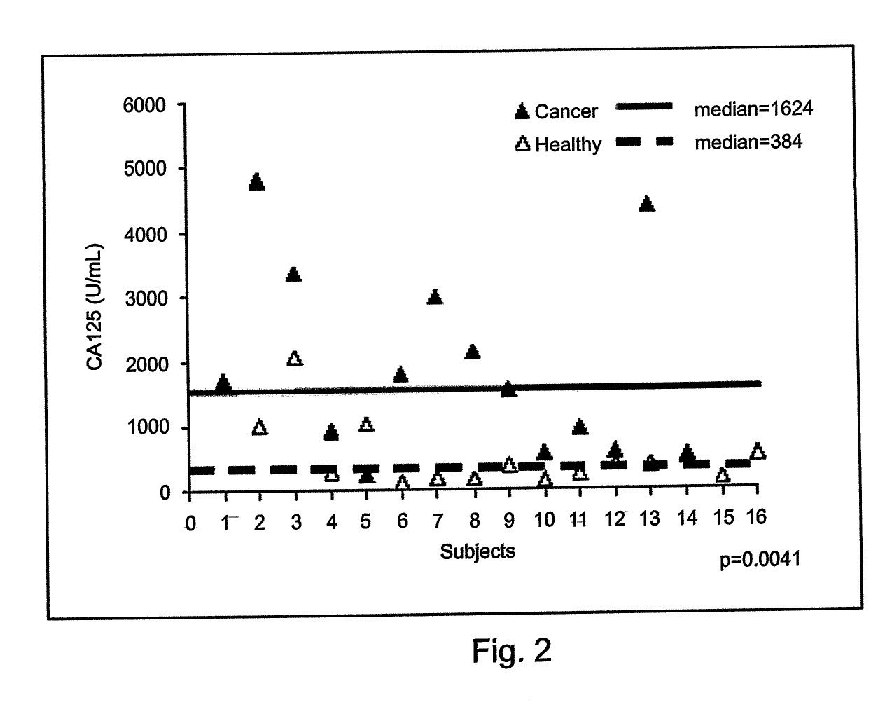

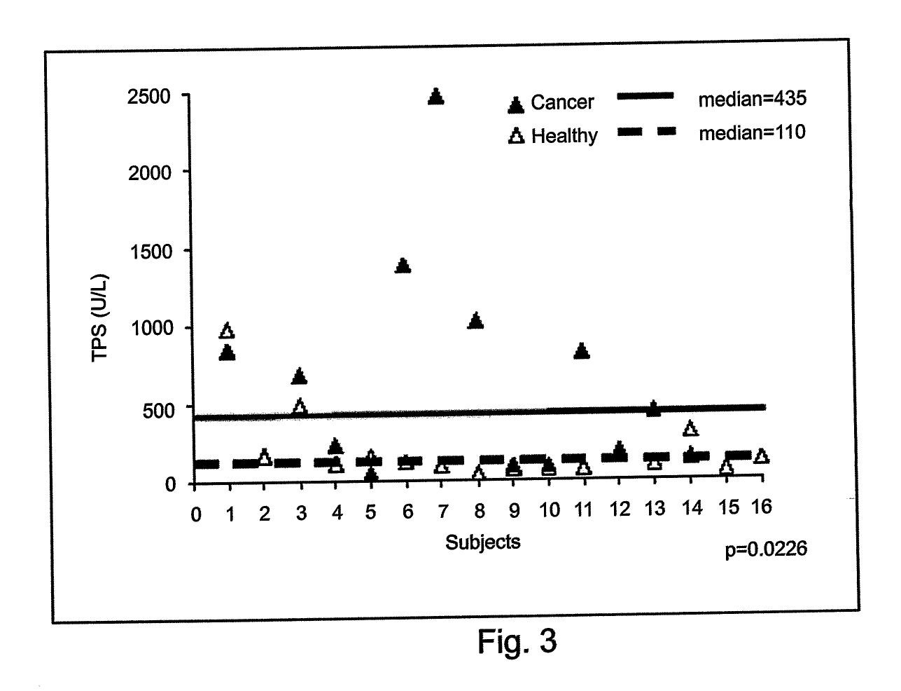

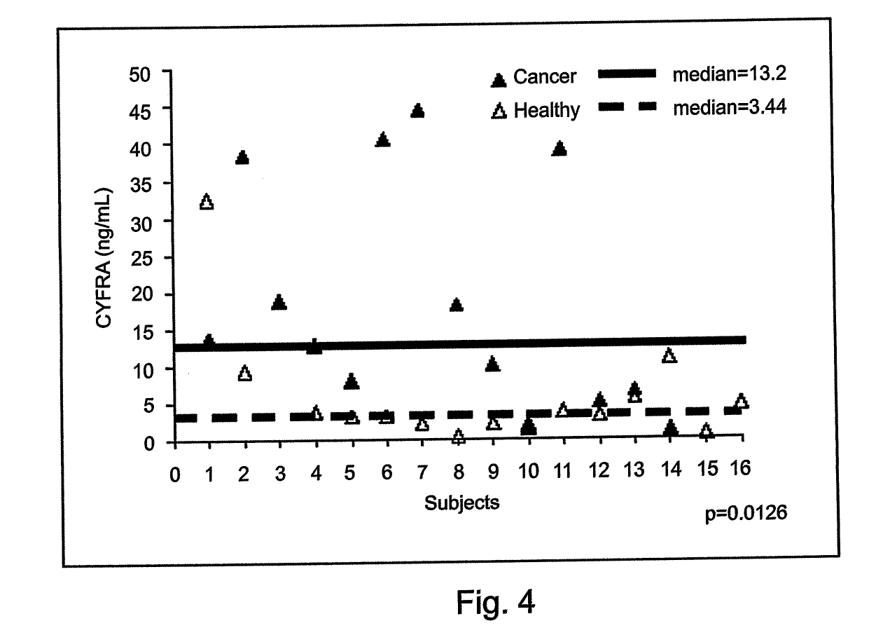

Individual analysis of salivary tumor markers: Salivary tumor marker analysis was available in 14 cancer patients and 16 healthy controls. The salivary concentrations in healthy control patients of CA125, TPS, Cyfra 21-1, CA19-9, CEA and SCC were 384 U/mL, 110 U/L, 3.44 ng/mL, 27.1 U/mL, 197.6 ng/mL and 140 ng/mL, respectively (FIGS. 1-4). The salivary concentrations of all six tumor markers were higher in cancer patients as compared to controls. The salivary levels in cancer patients of CA125, TPS, Cyfra 21-1, CA19-9, CEA and SCC were higher by 4.2 (p=0.0041), 3.9 (p=0.0026), 3.8 (p=0.0126), 2.9 (NS), 1.6 (NS) and 1.4 (NS) times as compared with controls, respectively (FIGS. 1-4). According to the matrix correlation analysis performed, a cross-talking among a few of these salivary markers was noted, i.e., a simultaneous increase of different markers in same patients. The correlation rate for Cyfra 21-1 and CA125 was 0.60 and for Cyfra 21-1 and TPS it was 0.90 while for Cyfra 21-1 and CEA it was 0.48 and for Cyfra 21-1 and SCC it was 0.45. The correlation rate for TPS and CEA was 0.70 and for TPS and CA125 it was 0.50.

Concurrent analysis of salivary tumor markers: The 3 salivary tumor markers which were found to be most substantially and significantly increased in the cancer patients were Cyfra 21-1, TPS and CA125, which were all increased by about 400%. Therefore an analysis was performed in which all patients in whom any of these three markers was equal or above cut-off levels were defined as patients with disease and vice-versa (̂ Disease: CA125≧1823 and/or Cyfra≧8.7 and/or TPS≧253, ̂ No-Disease: CA125<1823 and Cyfra<8.7 and TPS<253). The following values were found using this analysis:

1. Sensitivity-factor: (10/14)*100=71%

2. Specificity-factor: (12/16)*100=75%

3. Positive-predictive-value: (10/14)*100=71%

4. Negative-predictive-value: (12/16)*100=75%

5. False-negative-value: (4/14)*100=28%

6. False-positive-value: (4/16)*100=25%

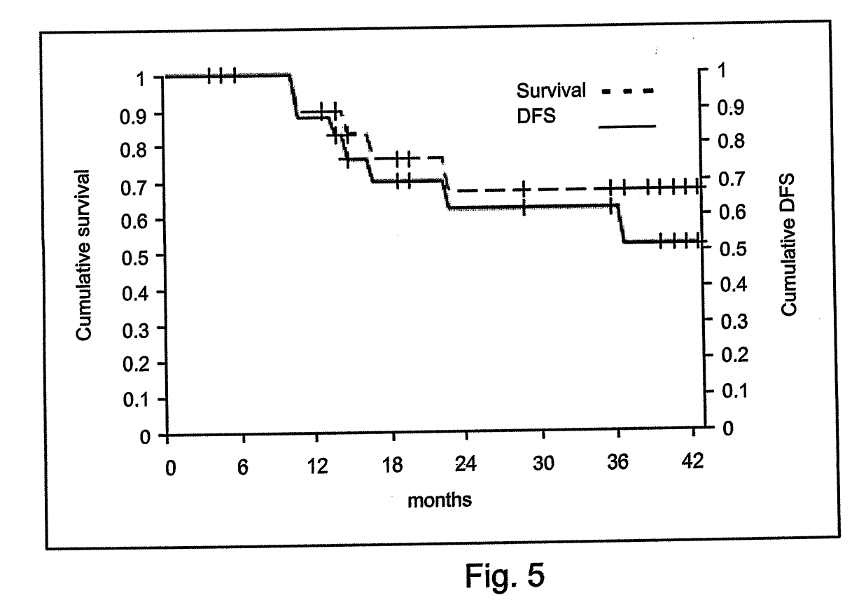

Cumulative survival: The cumulative survival rate of all patients (n=21) both at 24 and 36 months (two and three years) was 67%. The cumulative survival rate of all patients (n=21) at 42 months (3.5 years) was 67% (FIG. 5).

Cumulative Disease Free Survival (DFS): The cumulative DFS rate of all patients (n=21) at both 24 and 36 months (two and three years) was 63%. The cumulative DFS rate of all patients (n=21) at 42 months (3.5 years) was 52% (FIG. 5).

Cumulative DFS by grade: The cumulative DFS rate of patients with grade 1 at 42 months was 75%, higher than in patients with grade 2, which was 43%. The latter in turn was higher than in patients with grade 3 (0%). This difference was significant (p=0.05) as summarized in Table 1 herein below.

TABLE 1
Disease free survival (DFS) probability rates by
the pathological grading. p = 0.05 (Log-rank test).

| | | | | | DFS | DFS |
|---|---|---|---|---|---|---|
| DFS | Patients | Range | Died | Recurrence | at 24 | at 42 |
| Grade | (n = 21) | (months) | Patients | Patients | months | months |
| 1 | 7 | [14-43] | | 2 | 1.00 | 0.75 |
| 2 | 12 | [3-43] | 5 | | 0.43 | 0.43 |
| 3 | 2 | [3-13] | | 1 | 0 | 0 |

Cumulative DFS by stage: The cumulative DFS rate of patients with stage 1+2 at 42 months was 78%, significantly higher than in patients with stage 3+4 which was 17% (p=0.01), as summarized in Tables 2 and 3 herein below.

TABLE 2
Disease free survival (DFS) probability rates by staging:
1-4. p = 0.0273 (Log-rank test)

| | | | | | DFS | DFS |
|---|---|---|---|---|---|---|
| DFS | Patients | Range | Died | Recurrence | at | at 42 |
| Stage | (n = 21) | (months) | patients | patients | 24 months | months |
| 1 | 7 | [5-43] | 1 | 1 | 0.75 | 0.75 |
| 2 | 8 | [3-43] | | 1 | 0.83 | 0.83 |
| 3 | 4 | [10-36] | 3 | 1 | 0.25 | 0.0 |
| 4 | 2 | [14-41] | 1 | | 0.5 | 0.5 |

TABLE 3
Disease free survival (DFS) probability rates by staging:
1, 2 (early)-3, 4 (advanced). p = 0.01 (Log-rank test)

| | | | | | DFS | DFS |
|---|---|---|---|---|---|---|
| DFS | Patients | Range | Died | Recurrence | at | at 42 |
| Stage | (n = 21) | (months) | Patients | Patients | 24 months | months |
| 1-2 | 15 | [3-43] | 1 | 2 | 0.78 | 0.78 |
| 3-4 | 6 | [10-41] | 4 | 1 | 0.33 | 0.17 |

Cumulative DFS by N: The cumulative DFS rate of patients with N=0 at 42 months was 73%, higher than in patients with n=1 (25%). This difference did not reach statistical significance (p=0.14).

Cumulative DFS by site: The cumulative DFS rate of patients with base of tongue tumors (n=3) at 42 months was 55%, higher than in patients with mobile tongue tumors (n=18) which was 33%. This difference did not reach statistical significance (p=0.25).

Cumulative DFS by depth: The cumulative DFS rate of patients with depth<=5 mm was 75% was higher than in patients with depth>5 (23%). This differences did not reach statistical significance (p=0.13).

Cumulative DFS by diameter: The cumulative DFS rate of patients with diameter<2 cm, at 42 months was found higher than in patients with diameter≧2 cm (33%). This difference did not reach statistical significance (p=0.12).

Cumulative DFS by sex, age, smoking, other malignancies, extensiveness or salivary markers: The cumulative DFS rate of female patients at 42 months (72%) was found significantly higher than in male patients with (23%), (p=0.04). No significant correlations were found between the cumulative DFS values and any of the following parameters: age, smoking habits, other malignancies or an extension of the tumor beyond lingual margins. Positive correlations were not found between the cumulative DFS and any of the measured salivary marker levels.

Discussion

The most important result found was that several salivary tumor markers were found to be significantly increased (by 400%) in the saliva of oral (tongue) cancer patients. That is important with respect to both clinical and pathogenesis-related aspects of oral cancer and the various characteristics of this cancer show that indeed a representative group of tongue-cancer patients were analyzed in the current study. Both the total and the DFS survival probabilities were found to be similar to those found in other studies, as was the important predictive roles that tumor staging, grading, N and depth values have [Hinerman, R W. Head Neck 2004;26:984-94; Nagler et al., Cancer Lett 2002;186:137-50].

The increase in salivary tumor markers of the cancer patients may be used as a diagnostic tool, especially when a concurrent analysis is performed for several salivary markers. This suggests that this new diagnostic tool is of special importance for patient monitoring, as it is often very difficult to distinguish clinically between a post-operative and/or irradiated scarred oral mucosa and a recurring cancer lesion. Accordingly, such an analysis might turn into a valuable diagnostic tool as it might save many unnecessary biopsies and hospital/out patient clinic visits. Three of the markers analyzed (Cyfra 21-1, TPS and CA125) were significantly increased (by 400%, p≦0.01), while the increase of the other three did not reach statistical significance, probably resulting from a relatively large variation of the increase in these salivary tumor markers.

It was shown that when a concurrent analysis of the three significantly increased markers was performed, the sensitivity, specificity, negative and positive predictive values were in the range of 72-75%, comparable to those obtained when circulatory markers were measured in the serum of OSCC patients.

In summary, the significant increase in salivary tumor markers (approximately four-fold) is encouraging in light of the many advantages of saliva measurement in comparison with serum analysis. The definitive diagnosis of OSCC is obviously based on a harvested biopsy, but it would be highly desirable and beneficial if salivary tumor marker analysis could be performed on a routine basis between biopsies. The increase in salivary tumor markers may be used as a diagnostic tool, especially when a concurrent analysis for significantly increased markers is performed.

### Example 2

### Analysis of Oxidative Stress-Related Markers for the Detection of OSCC

Materials and Methods

Patients and study design: The patients were as for Example 1, herein above.

Saliva collection: Saliva was collected as described in Example 1.

Peroxidase Analysis: Peroxidase activity was measured both in the patients' serum and the saliva according to the NBS assay as previously described [Nagler and Reznick FRBM 2002,32:268-277]. Briefly, the calorimetric change induced by the reaction between the enzyme and the substrate, Dithiobis 2-Nitrobensoic Acid (DTNB) in the presence of mercapto-ethanol, was read at a wavelength of 412 nm for 20 seconds.

Glutathione S-transferase (GST) Analysis: The GST analysis was performed as previously described [Sundberg, Nephron, 1994;66(2):162-9]. Briefly, an enzyme-immuno assay (EIA) was employed allowing the quantitative determination of the human GST. The enzyme was first coated to the surface of microtiter plates followed by a blocking step and a pre-incubation of the calibrators and samples with a polyclonal rabbit antibody. The GST in the controls and samples then competed with the GST on the plate for antibody binding. After washing, the detection of the bound rabbit antibody was performed by peroxidase-labeled goat anti-rabbit antibody. The amount of converted substrate, indirectly proportional to the amount of GST antigen in the sample, was photometrically determined at 450 nm.

Superoxide dismutase (SOD) Analysis: Total activity of SOD isoenzymes (Cu\ Zn—SOD and Mn—SOD) was measured using the Xanthine Oxidase\XTT method. That is a spectrophotometric assay for SOD based on tetrazolium salt 3′-{1-[(phenylamino)-carbonyl]-3,4-tetrazolium}-bis(4-methoxy-6-nitro)benzenesulfonic acid hydrate reduction by xanthine-xanthine oxidase. The method is a modification of the NBT assay. Xtt is reduced by the superoxide anion (O₂⁻) generated by xanthine oxidase. Formazan is read at 470 nm. SOD inhibits this reaction by scavenging the O₂^{·}. One unit of the enzyme is defined as the amount of enzyme needed for 50% inhibition of absorption in the absence of the enzyme [Nagler, Free Radical Biology & Medicine. 32(3), 268-277 (2002)].

Uric acid (UA) concentration Analysis: Uric acid concentration was measured with a kit supplied by Sentinel CH (Milano, Italy) as previously described [Nagler, Free Radical Biology & Medicine. 32(3), 268-277 (2002)]. In the assay, uric acid is transformed by uricase into allantoin and hydrogen peroxide which, under the catalytic influence of peroxidase, oxidizes the chromogen (4-aminophenazone/N-ethyl-methylanilin propan-sulphonate sodic) to form a red compound whose intensity of color is proportional to the amount of uric acid present in the sample, and it is read at a wavelength of 546 nm.

Total antioxidant status (TAS) Analysis: The assay used was based on a commercial kit supplied by Randox (USA) in which metmyoglobin in the presence of iron is turned into ferrylmyoglobin. Incubation of the latter with the Randox reagent ABTS results in the formation of a blue-green colored radical which can be detected at 600 nm [Nagler, Free Radical Biology & Medicine. 32(3), 268-277 (2002)]).

Antioxidant capacity Analysis (InAnOx):An ELISA colorimetric test system (Immundiagnostik AG, Bensheim, Germany) for the determination of the overall antioxidative capacity of the oral cavity was performed by the reaction of antioxidants in saliva with a defined amount of exogenously provided hydrogen peroxide (H₂O₂). The antioxidants in the saliva sample eliminated a certain amount of the hydrogen peroxide provided. The residual H₂O₂ was determined colorimetrically by an enzymatic reaction which involves the conversion of TMB to a colored product. After the addition of a stop solution, the samples were measured at 450 nm in a microtiter plate reader. The quantification was performed by a calibrator. The difference between applied and measured concentration in a defined time is proportional to the reactivity of the antioxidants of the sample (antioxidant capacity).

Salivary nitrogen species analysis: Salivary nitric oxide (NO) was measured in terms of its products, nitrite (NO₂) and nitrate (NO₃), by the method of Griess modified by Fiddler [J Assoc Off Anal Chem. May 1997;60(3):594-9] using the Nitric Oxide and the Total Nitric Oxide assays kits of Assay Designs Inc. (Ann Arbor, Mich., USA). This method is based on a two-step process: the first step is the conversion of nitrate to nitrite using tin metal powder and the second is the addition of sulphanilamide and N(-naphthyl)ethylenediamine (Griess reagent). This converts nitrite into a deep purple azo compound, which was measured colorimetrically at 540 nm.

DNA analysis of salivary 8-hydroxy-deoxyguanosine (8-OHdG): Quantitative measurement of the oxidative DNA adduct 8-OHdG was performed according to the method described by Toyokuni et al. [Lab Invest. March 1997;76(3):365-74]. Briefly, the saliva samples were centrifuged at 10,000×g for 10 minutes, and the supernatant was used to determine 8-OHdG levels with a competitive ELISA kit (Japan Institute for the Control of Aging, Shizuoka, Japan). The determination range was 0.5-200 ng/ml.

Salivary carbonyls: Salivary carbonyls were analyzed by Western blot for both the healthy and OSCC groups being performed with Oxyblot Kit S-71250 (Intergen Co, NY, USA) using specific anti-Dinitrophenyhydrazine (DNPH) antibodies. Between 25 and 30 ml of saliva supernatant was applied to each well, corresponding to 60 mg of protein. Finally, saliva proteins were run on 10% SDS-PAGE (Polyacrylamide Gel Electrophoresis) as was described previously [Nagler and Lischinsky, J. Lab. Clin. Med. 137(5), 363-369 (2001)].

Statistical analysis: For categorical variables, frequencies, percentages and distribution were calculated. For continuous variables ranges and medians were calculated. Due to the large in-born variability of parameters in saliva and as is the common practice (Hardt), medians values were calculated and as small sample size groups analyzed (less then thirty individuals in a group) non-parametric statistical tests were used. Distributions of categorical variables were compared and analyzed by Fisher-Irwin exact test. The medians between subgroups of patients were compared by Wilcoxon rank-sum test (pairs of subgroups). The correlation between the parameters levels in patients and controls were analyzed with Spearman correlation.

Results

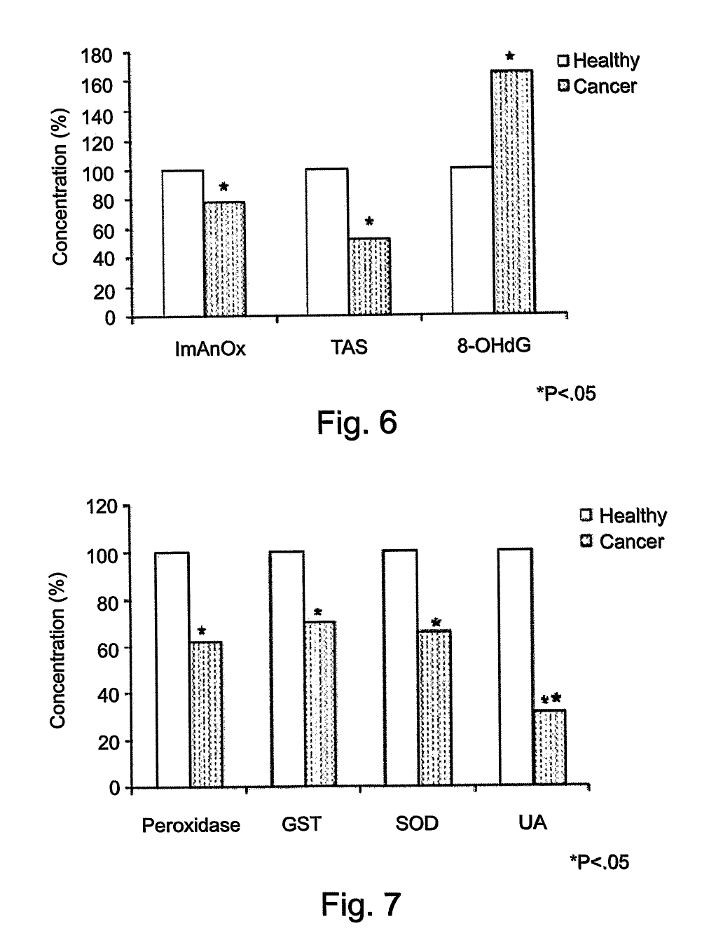

Antioxidant Analysis: Both ImAnOx and TAS assays evaluating the general antioxidant capacity of the saliva showed substantially reduced values in the OSCC patients as compared with controls (FIG. 6). The ImAnOx assay revealed a significantly reduced antioxidant capacity by 22% (P<0.05), from 320 mmol/L to 251 mmol/L, whereas the TAS assay revealed a 49% reduction, from 0.49 mmol/L to 0.25 mmol/L (P<0.05).

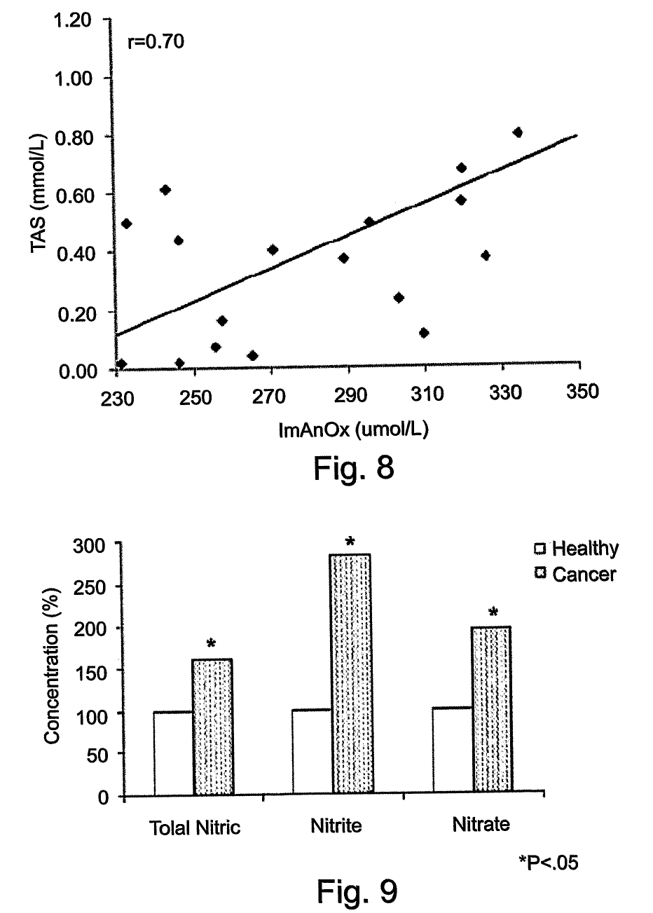

Similarly, the salivary-specific antioxidants analyzed (peroxidase, GST, and SOD enzymes and the UA molecule) were reduced by 38% (P<0.05), 30% (P<0.05), 34% (P<0.05), and 69% (P<0.01), respectively, from 386 mU/mL to 280 mU/mL, from 230 ng/mL to 161 ng/mL, from 1.25 U/mL to 0.90 U/mL, and from 4.12 mg/mL to 1.30 mg/mL (FIG. 7). Indeed, the Spearman correlation coefficients among the various analyzed antioxidants were rather high, indicating a similar pattern of reductions. Thus, the correlation coefficients of ImAnOx and peroxidase, ImAnOx and SOD, ImAnOx and UA, ImAnOx and TAS, and TAS and GST were 0.60, 0.55, 0.50, 0.70, and 0.55, respectively (FIG. 8).

Nitrogen Species Analysis: The salivary concentrations of the analyzed RNS: the NO, NO₂, and NO₃ in healthy controls, were 72 mmol/L, 80 mmol/L, and 37.6 mmol/L, respectively. In the OSCC patients these salivary values were higher by 60%, 190%, and 93%, respectively (P<0.05) (FIG. 9). The Spearman correlation coefficients between the NO and NO₂ and between the NO and NO₃ salivary concentrations were 0.90 and 0.66, respectively.

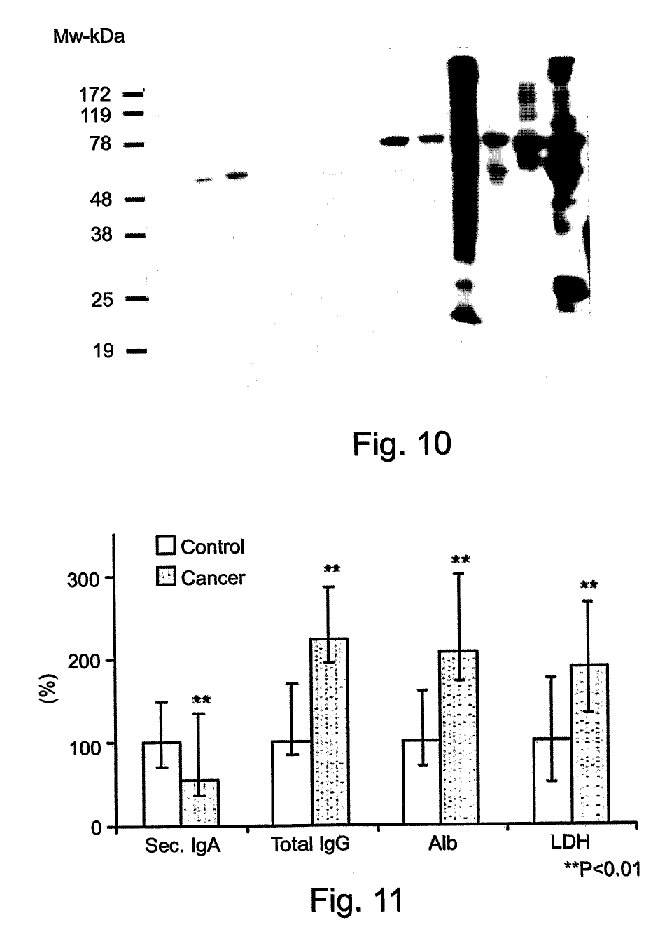

Oxidative DNA and Protein Analysis: The level of the oxidized DNA as expressed by 8-OHdG levels was increased by 65% (P<0.05) in the OSCC patients, from 0.68 ng/mL to 1.12 ng/mL (FIG. 10). The Spearman correlation coefficient between the 8-OHdG and ImAnOx was high as well (r ¼ 0.62). The Western blot presented in FIG. 9 clearly demonstrates the most extensive carbonylation level (indication of protein oxidation) in the saliva of OSCC patients as compared with controls.

Discussion

The novel and most interesting finding of the current study was that salivary composition of OSCC patients is substantially altered with respect to free radical-related mechanisms. The salivary DNA and proteins in these patients were found to be profoundly oxidized whereas all salivary RNS analyzed were found to be significantly increased and all salivary antioxidants significantly reduced.

The development of cancer is multifactorial, depending on the extent of DNA damage which is proportional to the magnitude of oxidative and nitrative stress. This stress reflects the net effect of both ROS and RNS on one hand and the effectiveness of antioxidant defense and the DNA repair systems on the other. In fact, it was found that while ROS and RNS are involved in the initiation and promotion of multistep carcinogenesis, both are inhibited by antioxidants [Sun, Free Radic Biol Med. 1990;8(6):583-99; Oberley, Mol Cell Biochem. December 1988;84(2):147-53]. However, when the equilibrium is broken either by a reduction in the levels of antioxidants or by enhancement of ROS and RNS levels, DNA is oxidized and cancer evolves. The present inventors observed this phenomenon in the saliva of the OSCC patients.

Nearly all the analyzed OSCC were of patients who belong to the de novo evolving cancer (“genetic”) group without a history of pre-malignant lesions or a history of smoking and drinking. Hence, in those patients hereditary predisposition factors are presumably responsible for the OSCC. It is tempting to speculate that in these patients some genetic factors (as an enhanced salivary transporter of nitrates or over-producing NO synthase (NOS) enzyme) are responsible for the increased levels of salivary RNS observed or perhaps vice versa, reduced activity of the salivary antioxidant enzyme\s or of the transport of UA. That is because in almost all the patients analyzed (all but 2 were non smokers), increased salivary RNS/ROS could not have originated from exposure to cigarette smoke or to any other known exogenous source.

### Example 3

### Biochemical and Immunological Analysis of Saliva for the Detection of OSCC

Materials and Methods

Patients and study design: The patients were as for Example 1, herein above.

Saliva collection: Saliva was collected as described in Example 1.

Biochemical and immunological analysis: The concentrations of the electrolytes Na and K were measured by Xame photometry, P concentration was measured spectophotometrically, and Ca and Mg concentrations were measured by atomic absorption as previously described [Baum et al. 1989, Amer J Physiol 246:35-39; Ben Aryeh et al. 1996, Biol Psychiatry 39:946-949]. Amy was measured by the Phadebas amylase test (Pharmacia Diagnostics, Uppsala, Sweden). Secretory IgA and Alb concentrations were measured by the radial-immunodiVusion method described by Mancini [Mancini et al. 1965, Immunochemistry 2:235-254], using an Oxford viewer for measuring the diameters of the precipitation rings. (The Mancini plates were purchased from Binding Side, Birmingham, UK.) The diameter of the ring formed is quantitatively related to the concentration of various parameters analyzed. Total IgG was determined by immunoturbidimetric methods on a Roche Cobas Mira automated analyzer using reagents purchased from Roche Diagnostics, Basel, Switzerland. LDH was measured at 37° C. by an optimized standard method using pyruvate as the substrate with the Hitachi 911 automated clinical chemistry analyzer using reagents purchased from Roche Diagnostics, Mannheim, Germany. The assay coefficient of variation (CV) was 2.1%. Amy was measured at 37° C. using 4,6-ethylidene (G7)-p-nitrophenyl (G1)-_,D-maltoheptaoside as substrate, as previously described [Hohenwaller et al. 1989, J Clin Chem Clin Biochem 27:97-101; Nagler et al. 2001, J Lab Clin Med 137:363-369]; the assay CV was 3.4%. IGF-I, EGF, MMP-2 and MMP-9 were measured by Quantikine solid phase ELISA kits (R&D Systems, Minneapolis, Minn., USA) (Bayes-Genis et al. 2000, Circ Res 86(2):125-130; McQuibban et al. 2000 Science 289(5482):1202-1206], as previously described.

Statistical analysis: For categorical variables, frequencies, percentages and distribution were calculated. For continuous variables, ranges and medians were calculated. Due to the large in-born variability of parameters in saliva and in accordance with common practice [Hardt et al. 2005, Anal Chem 77(15):4947-4954], median values were calculated and analyzed using non-parametric statistical tests, as is acceptable for small sample size groups (fewer than thirty individuals in a group). Distributions of categorical variables were compared and analyzed by Fisher-Irwin exact test. The medians between subgroups of patients were compared by Kruskal-Wallis (non-parametric multiple comparison test).

Results

Electrolytes, total protein and amylase: The salivary median TP concentration in the healthy control group was 68 mg/dl, while in the cancer patients it was significantly higher, by 26% (P=0.01). The salivary median Amy activity value in the healthy control group was 1,493 IU/l, while in the cancer patients it was lower in a non-significant manner, by 25% (P=0.12). Furthermore, the median salivary K concentration in the cancer patients was significantly lower (by 15%, P=0.03), while the concentrations of Na, Ca, P and Mg were higher in the saliva of the cancer patients by 14% (P=0.05), 59% (P=0.05), 39% (P=0.08) and 28% (P=0.12), respectively (Table 3, herein below).

TABLE 3
Salivary electrolytes, pH amylase (AMY) and total
protein (TP) composition in healthy controls
(n = 25) and OSCC patients (n = 25)

| | | | Differ- | Signifi- |
|---|---|---|---|---|
| | | | ence | cance |
| | Control | Cancer | (%) | (P) |
| pH (range) | (5.5-7.3) 6.4 | (5.8-7.3) 7 | +9 | 0.02* |
| median | | | | |
| Na (mmol/l), | (18-20) 19.8 | (20-37) 22.5 | +14 | 0.05* |
| (range) median | | | | |
| K (mmol/l), | (17.3-34.3) 24.3 | (15.6-30.0) 20.7 | −15 | 0.03* |
| (range) median | | | | |
| Ca (mg/dl), | (1.8-14.7) 3.7 | (3.3-7.9) 5.9 | +59 | 0.05* |
| (range) median | | | | |
| P (mg/dl), | (11.4-38.4) 16.8 | (8.9-47.4) 23.3 | +39 | 0.20 |
| (range) median | | | | |
| Mg (mg/dl), | (0.3-3.1) 0.7 | (0.4-2.0) 0.9 | +28 | 0.12 |
| (range) median | | | | |
| AMY (IU/l), | (388-4479) 1493 | (21-1988) 1125 | −25 | 0.12 |
| (range) median | | | | |
| TP (mg/dl), | (3177/57 | (3-215) 72 | +26 | 0.001** |
| (range) median | | | | |

Immunoglobulins, albumin and LDH: The salivary median concentration of Sec. IgA in the healthy control group was 599 mg/dl. In the cancer patients, this value was significantly lower by 45% (P=0.001). The salivary median concentration of total IgG in the healthy control group was 12.4 mg/dl. In the cancer patients, this value was significantly higher by 12% (P=0.01). The salivary median concentration of Alb in the healthy control group was 45 mg/dl, while in the cancer patients this value was higher by 108% (P=0.0007). The salivary median activity value of LDH in the healthy control group was 102 IU/l and 88% higher (P=0.002) (FIG. 11) in the cancer patients.

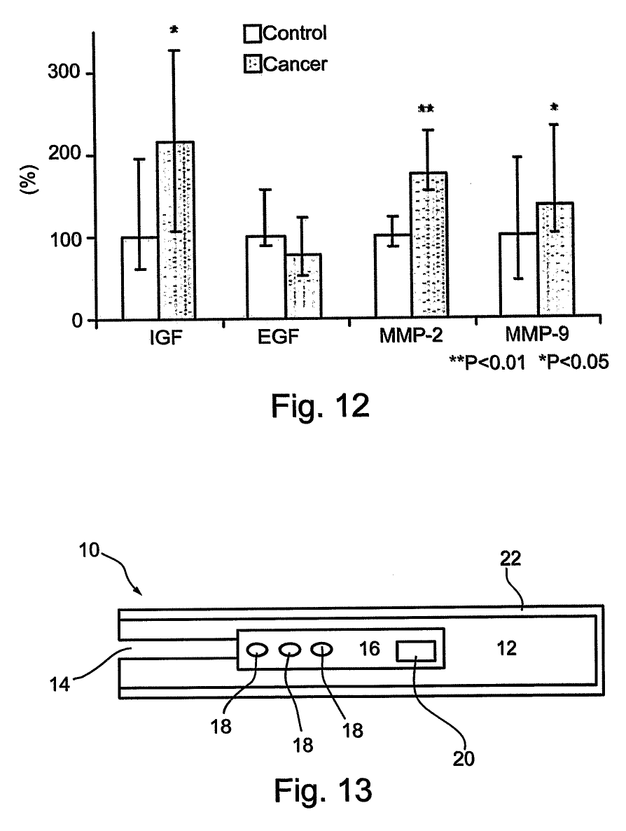

Growth factors and metalloproteases: The median salivary concentrations of secretory IGF, EGF, MMP-2 and MMP-9 of the control group were 0.17, 1.7, 3.1 and 427 ng/ml, respectively. In the cancer patients, the concentrations of IGF, MMP-2 and MMP-9 were significantly higher by 117% (P=0.03), 75% (P=0.0003) and 35% (P=0.05), respectively, while the EGF concentration was not significantly altered (FIG. 12). The sensitivity and specificity values of IGF, MMP-2 and MMP-9 were found to be in the range of 68-100. These values were calculated according to cut-off values that were computed as mean+standard error values and were 0.29, 3.77 and 493 ng/ml, respectively.

Discussion

The most interesting and novel finding of the current study was that a comprehensive salivary analysis revealed an overall altered salivary composition in OSCC. There were changes in almost all components evaluated, i.e., those which represented most of the salivary associated functional aspects and carcinogenesis-related factors. This indicates a compromised oral environment in oral cancer patients and sheds further light on the understanding of the disease pathogenesis. Moreover, these results may provide the clinician and/or the patient himself with an efficient, non-invasive and user-friendly new tool for OSCC diagnosis/monitoring.

For example, the altered concentrations of various salivary electrolytes and ions may compromise various salivary functions related to re-mineralization, maintaining buffering capacity, taste mediatory role etc., while reduced Amy activity may impair salivary digesting ability. Another interesting result relates to the increased concentration of total IgG, which indicates that the cancerous compromised oral mucosa is profoundly “leaking” serum-born ingredients such as IgG; this observation was further supported by the dramatic increase in salivary Alb (also a serum-born component) (Nagler et al. 2002, J. Invest Med 50(3) 214-225). This mutual increase in IgG and Alb is probably the major reason for the observed salivary protein increase.

In contrast to the IgG, the decrease observed in the concentration of Sec. IgA indicates local/regional changes. This occurrence may result either from a primary reduced antibacterial salivary capacity and/or an increased level of oral infections in the oral cavity of OSCC patients. Another result that seems to originate locally is the profound increase in salivary LDH. LDH is known to be mainly derived from exfoliative oral epithelial cells (in this case OSCC cells) and as such may also be used as a general salivary marker for the diseased mucosa.

As for the growth factors analyzed, it is interesting to note that, indeed, the concept was proved; i.e., there was a significant increase in IGF, MMP-2 and MMP-9. The increase in both growth factors and metalloproteinases points to their role in OSCC, as may be expected for epithelial cancer, which is in intimate continuous contact with the saliva that “bathes” the cancerous mucosa. MMP-2 and MMP-9 are metalloproteases that have been shown to participate in cancer pathogenesis as they degrade type-IV collagen, a major component of basement membrane, as well as other types of collagens (V, VII and X) and elastin and Fibronectin. They are highly expressed in stromal cells surrounding the invading front of metastasizing tumors and their levels are elevated in tumor endothelium and in urine of patients with various cancers (Fang et al. 2000, Proc Natl Acad Sci USA 97(8):3884-3889). Interestingly, the salivary IGF was increased most substantially (by 117%), while the EGF was not significantly altered. Both IGF and EGF are growth factors that have been shown to play a significant general role in carcinogenesis by modifying cancer-cell proliferation, survival, growth and apoptosis (Foulstone et al. 2005, J Pathol 205(2):145-153; Renehan et al. 2004, Lancet 363(9418):1346-1353), and both have been shown to interrelate in this process (Adams et al. 2004, Growth Factors 22(2):89-95; Kuribayashi et al. 2004, Endocrinology 145(11):4976-4984). Hence, it is very interesting to note the differentiated behavior of both in the present study, indicating IGF as the growth factor that plays an important role in OSCC pathogenesis.

### Example 4

### Diagnosing Oral Cancer by Detection of Saliva Secreted Markers

Materials and Methods

Parameters were measured in the saliva of cancer patients as described for Examples 1-3, herein above.

Statistical analysis: For categorical variables (sex), frequencies, percentages and distribution were calculated. For continuous variables ranges, medians, means and standard errors were calculated. Due to the large in-born variability of parameters in saliva, medians values, and as small sample size groups (less then thirty) non-parametric statistical tests were used. Distributions of categorical variables were compared and analyzed by “Fisher-Irwin exact test”. The medians between subgroups of subjects were compared by “Wilcoxon rank-sum test”. Correlation between pairs of variables was calculated by “Spearman correlation”. The ages between subgroups of subjects were compared by “Oneway analysis of variance”

Results

Uric/UA=uric acid

MMP=metalloproteinase

TAS=total antioxidant status

SOD=Superoxide dismutase

K=potassium

NA=sodium

CL=cloride

TP=Total protein

AM/Amy=Amylase

ALB=albumin

CA=calcium

B=PBR-binding.

PBR is the peripheral benzodiazepine receptor.

FR/Fr=flow rate (of saliva).

NS=non specific binding of the PBR

TP=total protein

LDH=lactate dehydrogenase

TABLE 4
Salivary markers in healthy controls and SCC patients

| | | Healthy | SCC | |
|---|---|---|---|---|
| | | (n = 18) | (n = 18) | p |
| CYFRA | Range | [2.03-31.49] | [0.83-8.11] | p = 0.98 |
| | Median | 3.58 | 6.05 | (NS) |
| CA19.9 | Range | [0.84-24002] | [4.9-53915] | p = 0.75 |
| (pg/ml) | Median | 627 | 538 | (NS) |
| CA125 | Range | [103.18-9550.4] | [80.84-1692.26] | p = 0.87 |
| | Median | 580 | 274 | (NS) |

TABLE 5
Salivary parameters in healthy controls and patients

| | | Healthy | SCC | |
|---|---|---|---|---|
| | | (n = 18) | (n = 18) | p |
| URIC | Range | [1.6-10.89] | [0.56-6.14] | p* = 0.0226 |
| mg/dl | Median | 4.45 | 2.53 | Sig |
| TAS | Range | [0.16-73.6] | [0.02-1.29] | p = 0.10 |
| mmol/L | Median | 0.495 | 0.25 | (NS) |
| SPO | Range | [0.157-1.00] | [0.0628-1.00] | p = 0.373 |
| O.D. | Median | 0.66 | 0.72 | (NS) |
| TP | Range | [23-159.4] | [5.6-298] | p = 0.85 |
| mg/dL | Median | 68.45 | 66.55 | (NS) |
| ALB | Range | [16.3-130.5] | [10.9-1060.7] | p = 0.338 |
| mg/dL | Median | 62.5 | 71.1 | (NS) |
| SOD | Range | [0.87-3.02] | [0.3-4.36] | p = 0.18 |
| U/mL | Median | 1.2 | 0.98 | (NS) |

TABLE 6
Age and gender distributions of healthy
controls and tongue cancer patients

| Gender | Obs. | (n = 35) | (n = 10) | |
|---|---|---|---|---|
| | Male | 20 (57%) | 4 (40%) | p = 0.34 |
| | Female | 15 (43%) | 6 (60%) | (NS) |
| age | Range | [70-86] | [70-81] | |
| | Mean | 76.85 | 77.00 | p = 0.94 |
| | St. dev. | 4.72 | 3.78 | (NS) |

TABLE 7
Age and gender distributions of healthy controls and tongue cancer patients

| | | Healthy (n = 35) | | Oral Cancer (n = 10) | p | |
|---|---|---|---|---|---|---|
| UA | Range | [0.95-10.77] | | [0.53-6.42] | | UA |
| | Mean | 4.90 | > | 3.30 | p = 0.0142 | |
| | St.err. | 0.35 | | 0.51 | Sig. | |
| SecIgA | Range | [188-1028] | | [153-630] | | SecIgA |
| | Mean | 599.40 | > | 384 | p = 0.0015 | |
| | St.err. | 33.50 | | 50.25 | Sig. | |
| K | Range | [16.6-37] | | [12.8-32.1] | | K |
| | Mean | 27.18 | > | 23.29 | p = 0.039 | |
| | St.err. | 1.10 | | 1.83 | Sig. | |
| G | Range | [0-2] | | [0-5] | | G |
| | Mean | 0.60 | < | 1.30 | p = 0.068 | |
| | St.err. | 0.15 | | 0.58 | (NS) | |
| SOD | Range | [307-1048] | | [480.4-804.6] | | SOD |
| | Mean | 586.65 | < | 648.77 | p = 0.12 | |
| | St.err. | 25.58 | | 35.50 | (NS) | |
| A | Range | [11-66] | | [11-60] | | A |
| | Mean | 27.30 | < | 31.80 | p = 0.20 | |
| | St.err. | 2.55 | | 5.26 | (NS) | |
| M | Range | [0-2] | | [0-0] | | M |
| | Mean | 0.62 | > | 0 | p = 0.16 | |
| | St.err. | 0.14 | | 0.00 | (NS) | |
| LYS | Range | [3-80] | | [4-35] | | LYS |
| | Mean | 23.40 | > | 15.30 | p = 0.13 | |
| | St.err. | 3.62 | | 3.50 | (NS) | |
| NA | Range | [4-33] | | [6-24] | | NA |
| | Mean | 11.31 | < | 13.60 | p = 0.14 | |
| | St.err. | 1.07 | | 1.70 | (NS) | |
| CL | Range | [18-45] | | [16-42] | | CL |
| | Mean | 28.83 | > | 28.20 | p = 0.40 | |
| | St.err. | 1.28 | | 1.52 | (NS) | |
| TP | Range | [10.6-199.4] | | [12.2-179.8] | | TP |
| | Mean | 93.40 | > | 82.77 | p = 0.27 | |
| | St.err. | 7.94 | | 16.92 | (NS) | |
| AM | Range | [320-3924] | | [568-2400] | | AM |
| | Mean | 1493.86 | > | 1125.60 | p = 0.12 | |
| | St.err. | 150.70 | | 214.44 | (NS) | |
| ALB | Range | [1.7-46.3] | | [1.5-25.5] | | ALB |
| | Mean | 13.80 | > | 11.61 | p = 0.28 | |
| | St.err. | 1.82 | | 2.87 | (NS) | |
| CA | Range | [1.1-20.6] | | [1.7-9.7] | | CA |
| | Mean | 4.34 | < | 5.79 | p = 0.13 | |
| | St.err. | 0.64 | | 0.80 | (NS) | |

TABLE 8
Bilinson tongue cancer 10 pts

| No | GrH1Ill2 | SOD | UA | A | G | M | SA | LYS |
|---|---|---|---|---|---|---|---|---|
| 81 | 2 | 560 | 2.33 | 11 | 0 | 0 | 153 | 8 |
| 82 | 2 | 712 | 2.16 | 22 | 0 | 0 | 250 | 5 |
| 84 | 2 | 611 | 3.58 | 45 | 1 | 0 | 466 | 18 |
| 85 | 2 | 791 | 2.88 | 32 | 1 | 0 | 345 | 8 |
| 86 | 2 | 805 | 0.53 | 17 | 0 | 0 | 200 | 4 |
| 87 | 2 | 715 | 2.89 | 21 | 0 | 0 | 419 | 10 |
| 88 | 2 | 520 | 4.51 | 60 | 5 | 0 | 580 | 30 |
| 89 | 2 | 480 | 6.42 | 55 | 2 | 0 | 630 | 35 |
| 90 | 2 | 591 | 3.05 | 34 | 4 | 0 | 480 | 25 |
| 91 | 2 | 703 | 4.66 | 21 | 0 | 0 | 317 | 10 |

| No | NA | K | CL | TP | ALB | CA | AM | AGE | M1F2 | NA |
|---|---|---|---|---|---|---|---|---|---|---|
| 81 | 14 | 20 | 22 | 12.2 | 5.6 | 6.4 | 568 | 79 | 2 | 14 |
| 82 | 15 | 19 | 24 | 19.6 | 4.2 | 4.4 | 808 | 70 | 1 | 15 |
| 84 | 11 | 26.2 | 30 | 109 | 10.2 | 3.1 | 870 | 81 | 1 | 11 |
| 85 | 6 | 25.7 | 27 | 97.3 | 4.4 | 1.7 | 690 | 79 | 1 | 6 |
| 86 | 9 | 12.8 | 16 | 34.8 | 1.5 | 4.3 | 1100 | 74 | 1 | 9 |
| 87 | 14 | 22.7 | 27 | 72 | 6.8 | 6 | 690 | 79 | 2 | 14 |
| 88 | 24 | 30.8 | 42 | 126 | 24.5 | 9.4 | 2400 | 77 | 2 | 24 |
| 89 | 14 | 32.1 | 38 | 121 | 22.2 | 6.2 | 1090 | | 2 | 14 |
| 90 | 20 | 24 | 34 | 180 | 25.5 | 9.7 | 2340 | | 2 | 20 |
| 91 | 9 | 19.6 | 22 | 55.9 | 11.2 | 6.7 | 700 | | 2 | 9 |

For the following Tables (9-14):

Subjects

| | |
|---|---|
| Gr1: Young non-smoking | 21 |
| Gr2: Old non-smoking | 12 |
| Gr3: Old smoking | 10 |
| Gr4: Tongue cancer | 8 |
| Total | 51 |

TABLE 9
Age by study groups

| Age | Gr1 | Gr2 | Gr3 | Gr4 | Gr1 + Gr2 + Gr3 |
|---|---|---|---|---|---|
| (year) | (n = 21) | (n = 12) | (n = 10) | (n = 8) | (n = 43) |
| range | [18-18] | [60-82] | [47-81] | [17-77] | [18-82] |
| mean | 18 | 70 | 57 | 52 | 41 |
| STD | 0 | 7.5 | 10.2 | 25 | 25 |

(p = 0.26) (oneway)

TABLE 10
Sex by study groups

| | Gr1 | Gr2 | Gr3 | Gr4 | Gr1 + Gr2 + Gr3 |
|---|---|---|---|---|---|
| Sex | (n = 21) | (n = 12) | (n = 10) | (n = 8) | (n = 43) |
| male | 15 (71%) | 4 (33%) | 5 (50%) | 3 (38%) | 23 (55%) |
| female | 6 (29%) | 8 (67%) | 5 (50%) | 5 (62%) | 19 (45%) |

TABLE 11

| | | Gr1 | Gr2 | Gr3 | Gr4 |
|---|---|---|---|---|---|
| | | (n = 21) | (n = 12) | (n = 10) | (n = 8) |
| Total | range | [1202-9842] | [1893-13530] | [2261-8333] | [4034-9176] |
| | median | 2745 | 6967 | 4031 | 6214 |
| | mean | 3292 | 7362 | 4328 | 6344 |
| | SEM | 440 | 935 | 585 | 760 |
| | p | Gr1 Vs. Gr2 | Gr2 Vs. Gr3 | | |
| | | p = 0.0008** | p = 0.02* | | |
| NS | range | [304-1520] | [441-2390] | [723-1879] | [716-3816] |
| | median | 739 | 1348 | 1046 | 1533 |
| | mean | 787 | 1363 | 1157 | 1792 |
| | SEM | 61 | 150 | 125 | 332 |
| | p | Gr1 Vs. Gr2 | Gr2 Vs. Gr3 | | |
| | | p = 0.0007** | p = 0.24 | | |
| B_ml | range | [898-8655] | [958-11633] | [1416-7216] | [2712-7293] |
| (mL) | median | 1959 | 5647 | 2579 | 4355 |
| | mean | 2504 | 6000 | 3170 | 4551 |
| | SEM | 415 | 820 | 589 | 570 |
| | p | Gr1 Vs. Gr2 | Gr2 Vs. Gr3 | | |
| | | p = 0.001** | p = 0.012* | | |
| B_TP | range | [261-5119] | [361-4373] | [411-1581] | [413-1258] |
| | median | 590 | 1045 | 615 | 528 |
| | mean | 875 | 1342 | 797 | 723 |
| | SEM | 228 | 310 | 126 | 123 |
| | p | Gr1 Vs. Gr2 | Gr2 Vs. Gr3 | | |
| | | p = 0.02* | p = 0.10 | | |
| FR | range | [0.08-0.495] | [0.05-0.225] | [0.22-0.675] | [0.30-0.67] |
| (ml/min) | median | 0.255 | 0.133 | 0.30 | 0.45 |
| | mean | 0.260 | 0.132 | 0.34 | 0.464 |
| | SEM | 0.02 | 0.01 | 0.04 | 0.05 |
| | p | Gr1 Vs. Gr2 | Gr2 Vs. Gr3 | | |
| | | p = 0.001** | p = 0.0001** | | |
| Protein | range | [0.036-0.536] | [0.007-0.80] | [0.09-0.309] | [0.164-0.627] |
| (mg/ml | median | 0.173 | 0.234 | 0.182 | 0.30 |
| saliva) | mean | 0.182 | 0.281 | 0.193 | 0.34 |
| | SEM | 0.024 | 0.068 | 0.02 | 0.05 |
| | p | Gr1 Vs. Gr2 | Gr2 Vs. Gr3 | | |
| | | p = 0.20 | p = 0.50 | | |
| B_ml/ | range | [2402-75260] | [5474-155106] | [2225-18502] | [4048-20433] |
| Fr | median | 8876 | 47752 | 9500 | 9163 |
| | mean | 12686 | 52450 | 9921 | 11460 |
| | SEM | 3424 | 10466 | 1683 | 2382 |
| | p | Gr1 Vs. Gr2 | Gr2 Vs. Gr3 | | |
| | | p = 0.0002** | p = 0.0005* | | |
| B_tp/ | range | [602-14626] | [3600-19435] | [1178-4583] | [616-4193] |
| Fr | median | 2893 | 8794 | 2255 | 1305 |
| | mean | 3898 | 9880 | 2423 | 1848 |
| | SEM | 805 | 1451 | 358 | 462 |
| | p | Gr1 Vs. Gr2 | Gr2 Vs. Gr3 | | |
| | | p = 0.0004** | p = 0.0001** | | |

**p ≦ 0.01;
*p ≦ 0.05;
NS p > 0.05;
(Wilcoxon ranksum test)

TABLE 12

| | | Gr1 + Gr2 + Gr3 | Gr4 |
|---|---|---|---|
| | | (n = 10) | (n = 8) |
| Total | range | [1202-13530] | [4034-9176] |
| | median | 3710 | 6214 |
| | mean | 4685 | 6344 |
| | SEM | 454 | 760 |
| | p | Gr1 + Gr2 + Gr3 Vs. | |
| | | Gr4 | |
| | | p = 0.048* | |
| NS | range | [304-2390] | [716-3816] |
| | median | 880 | 1533 |
| | mean | 1014 | 1792 |
| | SEM | 68 | 332 |
| | p | Gr1 + Gr2 + Gr3 Vs. | |
| | | Gr4 | |
| | | p = 0.009** | |
| B_ml | range | [898-11633] | [2712-7293] |
| (mL) | median | 2579 | 4355 |
| | mean | 3670 | 4551 |
| | SEM | 408 | 570 |
| | p | Gr1 + Gr2 + Gr3 Vs. | |
| | | Gr4 | |
| | | p = 0.107 | |
| B_TP | range | [261-5119] | [413-1258] |
| | median | 712 | 528 |
| | mean | 998 | 723 |
| | SEM | 149 | 123 |
| | p | Gr1 + Gr2 + Gr3 Vs. | |
| | | Gr4 | |
| | | p = 0.54 | |
| FR | range | [0.05-0.675] | [0.30-0.67] |
| (ml/min) | median | 0.24 | 0.45 |
| | mean | 0.24 | 0.464 |
| | SEM | 0.02 | 0.05 |
| | p | Gr1 + Gr2 + Gr3 Vs. | |
| | | Gr4 | |
| | | p = 0.0006** | |
| Protein | range | [0.007-0.80] | [0.164-0.627] |
| (mg/ml saliva) | median | 0.182 | 0.30 |
| | mean | 0.212 | 0.34 |
| | SEM | 0.02 | 0.05 |
| | p | Gr1 + Gr2 + Gr3 Vs. | |
| | | Gr4 | |
| | | p = 0.01** | |

**p ≦ 0.01;
*p ≦ 0.05;
NS p > 0.05; (Wilcoxon ranksum test)

TABLE 13

| | | Gr1 + Gr2 + Gr3 | Gr4 |
|---|---|---|---|
| | | (n = 10) | (n = 8) |
| B_ml/ | range | [2225-155107] | [4048-20433] |
| Fr | median | 10390 | 9163 |
| | mean | 23461 | 11460 |
| | SEM | 4427 | 2382 |
| p | p | Gr1 + Gr2 + Gr3 Vs. | |
| | | Gr4 | |
| | | p = 0.47 | |
| B_tp/ | range | [602-19435] | [616-4193] |
| Fr | median | 3639 | 1305 |
| | mean | 5289 | 1848 |
| | SEM | 735 | 462 |
| | p | Gr1 + Gr2 + Gr3 Vs. | |
| | | Gr4 | |
| | | p = 0.013* | |

**p ≦ 0.01;
*p ≦ 0.05;
NS p > 0.05; (Wilcoxon ranksum test)

TABLE 14
Correlation factor in young non-smoking subjects (n = 21)

| | | | | | | | Spearman |
|---|---|---|---|---|---|---|---|
| B_ml/Fr | Fr | Protein | B_tp | B_ml | NS | Total | corr. |
| | | | | | | 0.68 | NS |
| | | | | | 0.58 | 0.98 | B_ml |
| | | | | 0.57 | 0.25 | 0.55 | B_tp |
| | | | −0.35 | 0.52 | 0.33 | 0.53 | Protein |
| | | 0.01 | −0.20 | −0.18 | 0.08 | −0.12 | Fr |
| | −0.65 | 0.36 | 0.55 | 0.83 | 0.38 | 0.80 | B_ml/Fr |
| 0.80 | −0.75 | −0.25 | 0.76 | 0.50 | 0.10 | 0.45 | B_tp/Fr |

For the following Tables (15-17):

Subjects

| | |
|---|---|
| Gr2: Old non-smoking | 18 |
| Gr3: Old smoking | 16 |
| Gr4: Tongue cancer | 9 |
| Total | 51 |

TABLE 15

| | | Gr2 | Gr3 | Gr4 | p |
|---|---|---|---|---|---|
| pH | n | 18 | 16 | 9 | Gr 2/4 |
| | range | [5.5-7.3] | [5.5-7.3] | [5.8-7.3] | p = 0.15 |
| | median | 6.40 | 6.40 | 7.00 | Gr 3/4 |
| | mean | 6.58 | 6.34 | 6.87 | p = 0.027* |
| | SER | 0.12 | 0.13 | 0.17 | Gr 2/3 |
| | | | | | p = 0.19 |
| Na (mmol/L) | n | 12 | 15 | 9 | Gr 2/4 |
| | range | [20-20] | [17-20] | [20-37] | p = 0.09 |
| | median | 20 | 20 | 20 | Gr 3/4 |
| | mean | 20 | 19.8 | 22.22 | p = 0.05* |
| | SER | 0 | 0.20 | 1.88 | Gr 2/3 |
| | | | | | p = 0.37 |
| K (mmol/L) | n | 12 | 15 | 9 | Gr 2/4 |
| | range | [17.3-30.0] | [18.3-34.4] | [15.6-30.0] | p = 0.18 |
| | median | 25.2 | 24.2 | 20.7 | Gr 3/4 |
| | mean | 25.32 | 24.96 | 22.3 | p = 0.19 |
| | SER | 1.17 | 1.16 | 1.70 | Gr 2/3 |
| | | | | | p = 0.57 |
| Cl (mmol/L) | n | 12 | 15 | 15 | Gr 2/4 |
| | range | [20-45] | [20-41] | [20-69] | p = 0.83 |
| | median | 26 | 28 | 25 | Gr 3/4 |
| | mean | 27.25 | 28.0 | 32.1 | p = 0.70 |
| | SER | 2.16 | 1.53 | 5.56 | Gr 2/3 |
| | | | | | p = 0.54 |
| Ca (mg/dL) | n | 16 | 16 | 9 | Gr 2/4 |
| | range | [1.8-14.7] | [2.1-11.0] | [3.3-7.9] | p = 0.13 |
| | median | 3.55 | 3.85 | 5.9 | Gr 3/4 |
| | mean | 4.72 | 4.70 | 5.53 | p = 0.11 |
| | SER | 0.87 | 0.60 | 0.52 | Gr 2/3 |
| | | | | | p = 0.40 |
| P (mg/dL) | n | 16 | 16 | 9 | Gr 2/4 |
| | range | [11.4-38.4] | [9.3-32.6] | [8.9-47.4] | p = 0.19 |
| | median | 16.8 | 16.7 | 23.3 | Gr 3/4 |
| | mean | 18.4 | 18.27 | 24.67 | p = 0.20 |
| | SER | 1.63 | 1.70 | 4.05 | Gr 2/3 |
| | | | | | p = 0.80 |
| Mg (mg/dL) | n | 16 | 16 | 9 | Gr 2/4 |
| | range | [0.3-3.1] | [0.4-1.7] | [0.4-2.0] | p = 0.10 |
| | median | 0.70 | 0.70 | 0.90 | Gr 3/4 |
| | mean | 0.90 | 0.82 | 1.11 | p = 0.12 |
| | SER | 0.17 | 0.10 | 0.17 | Gr 2/3 |
| | | | | | p = 0.86 |

TABLE 16

| | | Gr2 | Gr3 | Gr4 | p |
|---|---|---|---|---|---|
| Fe (Ug/dL) | n | 16 | 16 | 2 | Gr 2/3 |
| | range | [0-12] | [0-2] | [1-2] | p = 0.70 |
| | median | 1.0 | 1.0 | 1.5 | |
| | mean | 2.62 | 1.13 | 1.5 | |
| | SER | 0.94 | 0.15 | 0.5 | |
| Li (mmol/L) | n | 15 | 15 | 8 | Gr 2/4 |
| | range | [0.1-0.25] | [0.02-0.21] | [0.1-0.31] | p = 0.74 |
| | median | 0.10 | 0.10 | 0.10 | Gr 3/4 |
| | mean | 0.12 | 0.11 | 0.15 | p = 0.39 |
| | SER | 0.01 | 0.01 | 0.03 | Gr 2/3 |
| | | | | | p = 0.45 |
| Uric (mg/dL) | n | 16 | 16 | 9 | Gr 2/4 |
| | range | [2.1-13.9] | [1.5-6.4] | [0-10.8] | p = 0.003** |
| | median | 4.15 | 4.10 | 1.30 | Gr 3/4 |
| | mean | 5.31 | 3.90 | 2.46 | p = 0.009** |
| | SER | 0.74 | 0.41 | 1.10 | Gr 2/3 |
| | | | | | p = 0.26 |
| TP (mg/dL) | n | 16 | 16 | 9 | Gr 2/4 |
| | range | [3-177] | [9-117] | [3-215] | p = 0.98 |
| | median | 74.5 | 63 | 66 | Gr 3/4 |
| | mean | 78.0 | 62.2 | 84.8 | p = 0.41 |
| | SER | 9.8 | 7.7 | 20.0 | Gr 2/3 |
| | | | | | p = 0.21 |
| ALB (mg/dL) | n | 16 | 16 | 9 | Gr 2/4 |
| | range | [12.5-352.2] | [6.8-79.0] | [109-771.4] | p = 0.0007** |
| | median | 65.75 | 24.15 | 139 | Gr 3/4 |
| | mean | 85.83 | 30.65 | 293.7 | p = 0.0001** |
| | SER | 22.24 | 5.80 | 86.0 | Gr 2/3 |
| | | | | | p = 0.003** |
| LDH (IU/L) | n | 16 | 13 | 9 | Gr 2/4 |
| | range | [11-554] | [2-217] | [22-563] | p = 0.28 |
| | median | 167 | 37 | 294 | Gr 3/4 |
| | mean | 232 | 61 | 297 | p = 0.002** |
| | SER | 44 | 21 | 56 | Gr 2/3 |
| | | | | | p = 0.003** |
| Amy (10\hat{ }² IU/L) | n | 17 | 16 | 9 | Gr 2/4 |
| | range | [388-4479] | [100-3663] | [21-1988] | p = 0.93 |
| | median | 1081 | 800 | 805 | Gr 3/4 |
| | mean | 1307 | 1230 | 1109 | p = 0.95 |
| | SER | 257 | 243 | 238 | Gr 2/3 |
| | | | | | p = 0.69 |
| IgA (mg/dL) | n | 9 | 9 | 5 | Gr 2/4 |
| | range | [13.5-29.7] | [14.6-21.3] | [10.7-49.9] | p = 0.07 |
| | median | 19.8 | 16.8 | 12.4 | Gr 3/4 |
| | mean | 20.8 | 16.8 | 19.6 | p = 0.07 |
| | SER | 1.82 | 0.67 | 7.6 | Gr 2/3 |
| | | | | | p = 0.07 |
| IgG (mg/dL) | n | 10 | 11 | 7 | Gr 2/4 |
| | range | [4.04-30.53] | [3.5-14.94] | [3.5-38.22] | p = 0.14 |
| | median | 11.07 | 4.31 | 28.06 | Gr 3/4 |
| | mean | 14.45 | 7.69 | 24.9 | p = 0.01** |
| | SER | 2.98 | 1.48 | 4.58 | Gr 2/3 |
| | | | | | p = 0.048* |

TABLE 17

| | | Gr2 | Gr3 | Gr4 | p |
|---|---|---|---|---|---|
| IgM | n | 11 | 11 | 7 | |
| (mg/dL) | range | [3.8-3.8] | [3.8-3.8] | [3.8-3.8] | |
| | median | 3.8 | 3.8 | 3.8 | |
| | mean | 3.8 | 3.8 | 3.8 | |
| | SER | 0 | 0 | 0 | |
| TAS | n | 8 | 16 | 9 | Gr 2/4 |
| (mmol/L) | range | [0.57-1.26] | [0.46-1.36] | [0.32-2.07] | p = 0.067 |
| | median | 0.80 | 0.88 | 0.56 | Gr 3/4 |
| | mean | 0.88 | 0.84 | 0.78 | p = 0.14 |
| | SER | 0.08 | 0.06 | 0.18 | Gr 2/3 |
| | | | | | p = 0.95 |
| SOD | n | 16 | 16 | 9 | Gr 2/4 |
| (U/mL) | range | [0.14-4.34] | [0.76-3.05] | [0.30-4.69] | p = 0.11 |
| | median | 1.05 | 1.38 | 1.47 | Gr 3/4 |
| | mean | 1.30 | 1.61 | 2.01 | p = 0.69 |
| | SER | 0.25 | 0.14 | 0.51 | Gr 2/3 |
| | | | | | p = 0.05* |
| SPO | n | 18 | 16 | 9 | Gr 2/4 |
| (O.D.) | range | [0.29-0.97] | [0.37-0.88] | [0.68-1.01] | p = 0.004** |
| | median | 0.80 | 0.78 | 0.94 | Gr 3/4 |
| | mean | 0.76 | 0.74 | 0.72 | p = 0.002** |
| | SER | 0.04 | 0.03 | 0.03 | Gr 2/3 |
| | | | | | p = 0.24 |
| Sec IgA | n | 10 | 16 | 9 | Gr 2/4 |
| (mG/L) | range | [136.6-686.1] | [126.8-696.2] | [114.3-1029.5] | p = 0.62 |
| | median | 277.5 | 222.0 | 174.2 | Gr 3/4 |
| | mean | 314.5 | 311.4 | 333.6 | p = 0.55 |
| | SER | 51.7 | 43.7 | | Gr 2/3 |
| | | | | | p = 0.80 |

For the following Tables (18-21):

| | |
|---|---|
| Gr1: Young non-smoking | 10 |
| Gr2: Old non-smoking | 10 |
| Gr3: Old smoking | 11 |
| Gr4: Tongue cancer | 9 |
| Total | 40 |

TABLE 18

| | | Gr1 | Gr2 | Gr3 | Gr4 |
|---|---|---|---|---|---|
| | | (n = 10) | (n = 10) | (n = 11) | (n = 9) |
| Prot. | range | [0.218-1.396] | [0.436-1.978] | [0.36-1.336] | [0.496-1.96] |
| Conc. | median | 0.481 | 0.635 | 0.604 | 0.72 |
| (mg/ml) | mean | 0.566 | 0.843 | 0.658 | 0.86 |
| | SEM | 0.10 | 0.166 | 0.08 | 0.15 |
| | p | Gr1 Vs. Gr2 | Gr2 Vs. Gr3 | | |
| | | p = 0.09 | p = 0.44 | | |
| MDA/ | range | [2.367-4.58] | [1.83-4.39] | [1.717-6.07] | [1.80-5.53] |
| TBARS | median | 3.70 | 3.02 | 2.82 | 3.93 |
| (nmol | mean | 3.50 | 3.04 | 3.44 | 3.83 |
| MDA/ml) | SEM | 0.23 | 0.29 | 0.47 | 0.44 |
| | p | Gr1 Vs. Gr2 | Gr2 Vs. Gr3 | | |
| | | p = 0.25 | p = 0.70 | | |
| carbonyls | range | [0.17-0.545] | [0.43-0.688] | [0.52-1.86] | [0.32-1.31] |
| (kit, | median | 0.29 | 0.54 | 1.22 | 0.36 |
| nmol/mg) | mean | 0.31 | 0.54 | 1.28 | 0.49 |
| | SEM | 0.04 | 0.02 | 0.11 | 0.10 |
| | p | Gr1 Vs. Gr2 | Gr2 Vs. Gr3 | | |
| | | p = 0.001** | p = 0.0006** | | |
| ImAnox | range | [273.9-371.2] | [342.7-393.8] | [387.6-392] | [269.5-396.2] |
| (umol/l) | median | 349.7 | 385.9 | 390.5 | 318.2 |
| | mean | 337.3 | 378.6 | 390.5 | 329.8 |
| | SEM | 9.46 | 5.3 | 0.35 | 15.68 |
| | p | Gr1 Vs. Gr2 | Gr2 Vs. Gr3 | | |
| | | p = 0.001** | p = 0.066 | | |
| Nitrate | range | [49.62-259.3] | [153.95-1065] | [17.31-702.1] | [9.29-472.3] |
| (umol/l) | median | 93.73 | 595.5 | 135.2 | 72.24 |
| | mean | 126.48 | 573.6 | 233.9 | 123.1 |
| | SEM | 23.42 | 105.2 | 66.98 | 50.15 |
| | p | Gr1 Vs. Gr2 | Gr2 Vs. Gr3 | | |
| | | p = 0.001** | p = 0.01* | | |
| nitrite | range | [52.19-288.87] | [166.4-1189.6] | [19.4-777.75] | [6.27-484.6] |
| (umol/l) | median | 99.26 | 780.75 | 147.2 | 70.47 |
| | mean | 135.4 | 675.72 | 260.66 | 125.6 |
| | SEM | 26.36 | 124.6 | 75.0 | 52.74 |
| | p | Gr1 Vs. Gr2 | Gr2 Vs. Gr3 | | |
| | | p = 0.001** | p = 0.008** | | |

**p ≦ 0.01;
*p ≦ 0.05;
NS p > 0.05;
(Wilcoxon ranksum test)

TABLE 19

| | | Gr1 + Gr2 + Gr3 | Gr4 |
|---|---|---|---|
| | | (n = 10) | (n = 8) |
| Prot. | range | [0.218-1.978] | [0.496-1.96] |
| Conc. | median | 0.572 | 0.72 |
| (mg/ml) | mean | 0.69 | 0.86 |
| | SEM | 0.07 | 0.15 |
| | p | Gr1 + Gr2 + Gr3 Vs. | |
| | | Gr4 | |
| | | p = 0.01** | |
| MDA/ | range | [1.72-6.07] | [1.80-5.53] |
| TBARS (nmol | median | 3.13 | 3.93 |
| MDA/ml) | mean | 3.33 | 3.83 |
| | SEM | 0.20 | 0.44 |
| | p | Gr1 + Gr2 + Gr3 Vs. | |
| | | Gr4 | |
| | | p = 0.30 | |
| carbonyls (kit, | range | [0.17-1.86] | [0.32-1.31] |
| nmol/mg) | median | 0.55 | 0.36 |
| | mean | 0.73 | 0.49 |
| | SEM | 0.09 | 0.10 |
| | p | Gr1 + Gr2 + Gr3 Vs. | |
| | | Gr4 | |
| | | p = 0.12 | |
| ImAnox (umol/l) | range | [273.9-393.9] | [269.5-396.2] |
| | median | 387.6 | 318.2 |
| | mean | 369.5 | 329.8 |
| | SEM | 5.35 | 15.68 |
| | | Gr1 + Gr2 + Gr3 Vs. | |
| | p | Gr4 | |
| | | p = 0.02* | |
| Nitrate (umol/l) | range | [17.3-1065] | [9.29-472.3] |
| | median | 186.1 | 72.24 |
| | mean | 308.9 | 123.1 |
| | SEM | 53.2 | 50.15 |
| | p | Gr1 + Gr2 + Gr3 Vs. | |
| | | Gr4 | |
| | | p = 0.02* | |
| nitrite (umol/l) | range | [19.4-1189.6] | [6.27-484.6] |
| | median | 203.4 | 70.47 |
| | mean | 354.1 | 125.6 |
| | SEM | 63.0 | 52.74 |
| | p | Gr1 + Gr2 + Gr3 Vs. | |
| | | Gr4 | |
| | | p = 0.017* | |

**p ≦ 0.01;
*p ≦ 0.05;
NS p > 0.05; (Wilcoxon ranksum test)

TABLE 20

| | | Gr1 | Gr2 | Gr3 | Gr4 |
|---|---|---|---|---|---|
| | | (n = 10) | (n = 10) | (n = 11) | (n = 9) |
| tot. nit. Ox. | range | [4.88-27.56] | [16.07-116.97] | [1.72-76.87] | [0.63-95.58] |
| (umol/l) | median | 9.59 | 73.94 | 14.35 | 7.02 |
| | mean | 13.12 | 66.93 | 25.31 | 17.83 |
| | SEM | 2.53 | 12.71 | 7.37 | 10.08 |
| | p | Gr1 Vs. Gr2 | Gr2 Vs. Gr3 | | |
| | | p = 0.001** | p = 0.008** | | |
| MMP2 | range | [1.83-4.73] | [2.53-11.56] | [2.26-4.57] | [4.086-12.37] |
| (ng/ml) | median | 2.98 | 4.00 | 2.58 | 5.10 |
| | mean | 3.08 | 4.58 | 2.76 | 6.14 |
| | SEM | 0.32 | 0.83 | 0.20 | 0.86 |
| | p | Gr1 Vs. Gr2 | Gr2 Vs. Gr3 | | |
| | | p = 0.075 | p = 0.005** | | |
| MMP9 | range | [104.17-784.1] | [77.65-1490.5] | [96.59-962.12] | [217.8-1585.2] |
| (ng/ml) | median | 307.77 | 672.35 | 304.92 | 456.44 |
| | mean | 350.2 | 658.52 | 304.92 | 633.63 |
| | SEM | 73.03 | 125.85 | 74.78 | 143.42 |
| | p | Gr1 Vs. Gr2 | Gr2 Vs. Gr3 | | |
| | | p = 0.07 | p = 0.035* | | |
| Heparanase | range | [0-767.86] | [67.86-525] | [0-1246.43] | [17.86-582.14] |
| | median | 10.71 | 160.7 | 26.79 | 321.43 |
| | mean | 132.9 | 228.06 | 341.96 | 333.93 |
| | SEM | 86.0 | 62.93 | 166.0 | 69.2 |
| | p | Gr1 Vs. Gr2 | Gr2 Vs. Gr3 | | |
| | | p = 0.048* | p = 0.45 | | |
| Total | range | [0.218-1.396] | [0.436-1.98] | [0.36-1.336] | [0.496-1.96] |
| | median | 0.481 | 0.635 | 0.604 | 0.72 |
| | mean | 0.566 | 0.84 | 0.658 | 0.86 |
| | SEM | 0.10 | 0.17 | 0.08 | 0.15 |
| | p | Gr1 Vs. Gr2 | Gr2 Vs. Gr3 | | |
| | | p = 0.10 | p = 0.44 | | |
| 20 mg/total | range | [14.33-91.74] | [10.11-45.87] | [14.97-55.56] | [10.20-40.32] |
| | median | 41.78 | 31.52 | 33.11 | 27.78 |
| | mean | 43.89 | 30.06 | 34.67 | 27.43 |
| | SEM | 6.54 | 3.89 | 3.64 | 3.32 |
| | p | Gr1 Vs. Gr2 | Gr2 Vs. Gr3 | | |
| | | p = 0.10 | p = 0.44 | | |
| DDW | range | [108.26-185.7] | [154.13-189.9] | [144.44-185.0] | [159.68-189.8] |
| (200ul) | median | 158.22 | 168.48 | 166.89 | 172.22 |
| | mean | 156.11 | 169.94 | 165.33 | 172.57 |
| | SEM | 6.54 | 3.90 | 3.64 | 3.32 |
| | p | Gr1 Vs. Gr2 | Gr2 Vs. Gr3 | | |
| | | p = 0.10 | p = 0.44 | | |

**p ≦ 0.01;
*p ≦ 0.05;
NS p > 0.05;
(Wilcoxon ranksum test)

TABLE 21

| | | Gr1 + Gr2 + Gr3 | Gr4 |
|---|---|---|---|
| | | (n = 10) | (n = 8) |
| tot. nit. Ox. | range | [1.715-116.97] | [0.63-95.58] |
| (umol/l) | median | 19.32 | 7.02 |
| | mean | 34.80 | 17.83 |
| | SEM | 6.32 | 10.08 |
| | p | Gr1 + Gr2 + Gr3 Vs. | |
| | | Gr4 | |
| | | p = 0.027* | |
| MMP2 | range | [1.83-11.56] | [4.086-12.37] |
| (ng/ml) | median | 2.90 | 5.10 |
| | mean | 3.45 | 6.14 |
| | SEM | 0.32 | 0.86 |
| | p | Gr1 + Gr2 + Gr3 Vs. | |
| | | Gr4 | |
| | | p = 0.0003** | |
| MMP9 | range | [77.65-1490.5] | [217.8-1585.2] |
| (ng/ml) | median | 337.12 | 456.44 |
| | mean | 433.59 | 633.63 |
| | SEM | 59.37 | 143.42 |
| | p | Gr1 + Gr2 + Gr3 Vs. | |
| | | Gr4 | |
| | | p = 0.113 | |
| Heparanase | range | [0-1246.43] | [17.86-582.14] |
| | median | 110.71 | 321.43 |
| | mean | 230.36 | 333.93 |
| | SEM | 66.32 | 69.2 |
| | p | Gr1 + Gr2 + Gr3 Vs. | |
| | | Gr4 | |
| | | p = 0.05* | |
| Total | range | [0.218-1.978] | [0.496-1.96] |
| | median | 0.572 | 0.72 |
| | mean | 0.69 | 0.86 |
| | SEM | 0.07 | 0.15 |
| | p | Gr1 + Gr2 + Gr3 Vs. | |
| | | Gr4 | |
| | | p = 0.10 | |
| 20 mg/total | range | [10.11-91.74] | [10.20-40.32] |
| | median | 34.97 | 27.78 |
| | mean | 36.16 | 27.43 |
| | SEM | 2.87 | 3.32 |
| | p | Gr1 + Gr2 + Gr3 Vs. | |
| | | Gr4 | |
| | | p = 0.10 | |
| DDW | range | [108.56-189.89] | [159.68-189.8] |
| (200 ul) | median | 165.04 | 172.22 |
| | mean | 163.84 | 172.57 |
| | SEM | 2.87 | 3.32 |
| | p | Gr1 + Gr2 + Gr3 Vs. | |
| | | Gr4 | |
| | | p = 0.10 | |

**p ≦ 0.01;
*p ≦ 0.05;
NS p > 0.05; (Wilcoxon ranksum test)

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the present invention.

1. A method of diagnosing cancer in a subject, the method comprising determining a level and/or activity of at least one saliva secreted marker in a saliva sample of the subject wherein an alteration in said marker with respect to an unaffected saliva sample is indicative of the cancer, with the proviso that the saliva secreted marker is not circulatory carcinoembryonic antigen (CEA).

2. A method of diagnosing cancer in a subject, the method comprising determining a level and/or activity of at least one marker in a saliva sample of the subject wherein an alteration in said marker with respect to an unaffected saliva sample is indicative of the cancer, wherein said saliva marker is selected from the group consisting of tissue polypeptide-specific antigen (TPS), Cyfra 21-1, 8-Hydroxy-2′-deoxyguanosine (8OHDG), Squamous cell carcinoma (SCC) antigen, CA19-9, CA125, a free radical, a nitrate, a nitrite, a nitric oxide, a carbonyl polypeptide, a thiobarbituric acid reactive substance (TBARS), malondialdehyde (MDA), glutathione S-transferase (GST), Superoxide dismutase (SOD), Uric acid (UA), Ferrylmyoglobin, total antioxidant status (TAS), peroxidase, antioxidant capacity (ImAnOx), Metalloproteinase, Benzodiazepine receptor, pH, Heparanase, total protein, amylase, an electrolyte, lactate dehydrogenase (LDH), insulin-like growth factor (IGF), epidermal growth factor (EGF) and albumin.

3. The method of claim 1, wherein said saliva secreted marker is selected from the group consisting of TPS, Cyfra 21-1, Squamous cell carcinoma (SCC) antigen, CA19-9, CA125, a free radical, a nitrate, a nitrite, a nitric oxide, a carbonyl polypeptide, a thiobarbituric acid reactive substance (TBARS), malondialdehyde (MDA), glutathione S-transferase (GST), Superoxide dismutase (SOD), 8-Hydroxy-2′-deoxyguanosine (8OHDG), Uric acid, Ferrylmyoglobin, peroxidase, Metalloproteinase, Benzodiazepine receptor, Heparanase, total protein, amylase, an electrolyte, lactate dehydrogenase (LDH), insulin-like growth factor (IGF), epidermal growth factor (EGF) and albumin.

4. The method of claim 1, wherein said saliva secreted marker is selected from the group consisting of a tumor marker, a reactive nitrogen species, a reactive oxygen species and an antioxidant marker.

5. The method of claim 4, wherein said reactive oxygen species is selected from the group consisting of a superoxide radical (O₂⁻), a hydroxyl radical (OH⁻), and hydrogen peroxide (H₂O₂).

6. The method of claim 4, wherein said reactive nitrogen species is selected from the group consisting of a nitrate, a nitrite and nitric oxide.

7. The method of claim 4, wherein said tumor marker is selected from the group consisting of TPS, Cyfra 21-1, SCC, CA19-9 and CA125.

8. The method of claim 1, wherein said saliva secreted marker is a polypeptide or a fatty acid.

9. The method of claim 8, wherein said polypeptide is a carbonyl polypeptide.

10. The method of claim 8, wherein said fatty acid is MDA or TBARS.

11. The method of claim 4, wherein said antioxidant marker is selected from the group consisting of GST, SOD, Uric acid, ferrylmyoglobin and a peroxidase.

12. The method of claim 2, further comprising determining a level of CEA in said saliva sample.

13. The method of claim 1, wherein said cancer is oral cancer or oral-pharyngeal cancer.

14. A kit for diagnosing cancer in a subject, the kit comprising a packaging material which comprises at least one agent for specifically determining a level and/or activity of at least one saliva secreted marker in a saliva sample of the subject, with the proviso that said saliva secreted marker is not CEA.

15. A kit for diagnosing cancer in a subject the kit comprising a packaging material which comprises at least one agent for specifically determining a level and/or activity of at least one saliva marker in a saliva sample of the subject, said saliva marker being selected from the group consisting of tissue polypeptide-specific antigen (TPS), Cyfra 21-1, 8-Hydroxy-2′-deoxyguanosine (8OHDG), Squamous cell carcinoma (SCC) antigen, CA19-9, CA125, a free radical, a nitrate, a nitrite, a nitric oxide, a carbonyl polypeptide, a thiobarbituric acid reactive substance (TBARS), malondialdehyde (MDA), glutathione S-transferase (GST), Superoxide dismutase (SOD), Uric acid (UA), Ferrylmyoglobin, total antioxidant status (TAS), peroxidase, antioxidant capacity (ImAnOx), Metalloproteinase, Benzodiazepine receptor, pH, Heparanase, total protein, amylase, an electrolyte, lactate dehydrogenase (LDH), insulin-like growth factor (IGF), epidermal growth factor (EGF) and albumin.

16. The kit of claim 14, wherein said saliva secreted marker is selected from the group consisting of TPS, Cyfra 21-1, Squamous cell carcinoma (SCC) antigen, CA19-9, CA125, a free radical, a nitrate, a nitrite, a nitric oxide, a carbonyl polypeptide, a thiobarbituric acid reactive substance (TBARS), malondialdehyde (MDA), glutathione S-transferase (GST), Superoxide dismutase (SOD), 8-Hydroxy-2′-deoxyguanosine (8OHDG), Uric acid, Ferrylmyoglobin, peroxidase, Metalloproteinase, Benzodiazepine receptor, Heparanase, total protein, amylase, an electrolyte, lactate dehydrogenase (LDH), insulin-like growth factor (IGF), epidermal growth factor (EGF) and albumin.

17. The kit of claim 14, wherein said saliva secreted marker is selected from the group consisting of a tumor marker, a reactive nitrogen species marker, a reactive oxygen species marker and an antioxidant marker.

18.-20. (canceled)

21. The kit of claim 14, wherein said saliva secreted marker is a polypeptide or a fatty acid.

22.-24. (canceled)

25. The kit of claim 15, further comprising at least one agent for specifically determining a level and/or activity of CEA.

26. A device for diagnosing cancer, the device comprising a support and at least one agent for specifically determining a level and/or activity of at least one saliva marker in a saliva sample of the subject attached to said support, said saliva marker being selected from the group consisting of tissue polypeptide-specific antigen (TPS), Cyfra 21-1, 8-Hydroxy-2′-deoxyguanosine (8OHDG), Squamous cell carcinoma (SCC) antigen, CA19-9, CA125, a free radical, a nitrate, a nitrite, a nitric oxide, a carbonyl polypeptide, a thiobarbituric acid reactive substance (TBARS), malondialdehyde (MDA), glutathione S-transferase (GST), Superoxide dismutase (SOD), Uric acid (UA), Ferrylmyoglobin, total antioxidant status (TAS), peroxidase, antioxidant capacity (ImAnOx), Metalloproteinase, Benzodiazepine receptor, pH, Heparanase, total protein, amylase, an electrolyte, lactate dehydrogenase (LDH), insulin-like growth factor (IGF), epidermal growth factor (EGF) and albumin.

27. The device of claim 26, wherein said at least one agent is an antibody.

28. The device of claim 26, being a lateral flow device.

29. The device of claim 26, being a dipstick or a cartridge.

30. The method of claim 2, wherein said cancer is oral cancer or oral-pharyngeal cancer.
